(19) United States
(12) Patent Application Publication
(10) Pub. No.: US 2023/0142705 A1
(43) Pub. Date: May 11, 2023

(54) USE OF ANTI-AGING GLYCOPEPTIDES FOR INHIBITION OF IMMUNE REJECTION OF A GRAFT

(71) Applicant: PROTOKINETIX INC., Marietta, OH (US)

(72) Inventor: Lachlan Grant YOUNG, Nanaimo (CA)

(21) Appl. No.: 17/793,516

(22) PCT Filed: Jan. 22, 2021
(86) PCT No.: PCT/CA2021/050068

§ 371 Date: Jul. 18, 2022

Related U.S. Application Data
(60) Provisional application No. 62/965,289, filed on Jan. 24, 2020.
(60) Provisional application No. 63/118,715, filed on Nov. 26, 2020.

(51) Int. Cl. A61K 38/17; A61P 37/06; C12N 5/0797

(52) U.S. Cl. CPC A61K 38/1706; A61P 37/06; C12N 5/0623; C12N 2500/33; C12N 2501/105; C12N 2506/02

(57) ABSTRACT

The present document describes uses and methods of using a gem-difluorinated C-glycopeptide compound of general formula I, or a pharmaceutically acceptable base, addition salt with an acid, hydrate or solvate of the compound of general formula I for inhibition or prevention of immune rejection of an isolated graft contacted with said compound, prior to transplantation in a subject in need thereof.

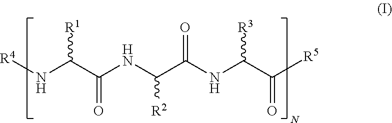

### CROSS-REFERENCE TO RELATED APPLICATIONS

This application claims priority of US provisional patent applications Nos. 62/965,289 filed on Jan. 24, 2020, and 63/118,715, filed on Nov. 26, 2020, the specifications of which are hereby incorporated by reference in their entireties.

### BACKGROUND

### (a) Field

The subject matter disclosed generally relates to a method of transplantation of a graft, and more particularly to a method for the inhibition or prevention of immune rejection against a graft transplanted in a subject in need of transplantation in which immunosuppressant drug therapy has been discontinued after transplantation.

### (b) Related Prior Art

Antifreeze biological compounds, and particularly glycoproteins, exist in the natural environment. These compounds are present for example in some fishes, enabling them to survive in a low temperature environment (i.e. near zero or sub-zero temperatures). Scientists have been investigating how antifreeze compounds taken from the natural environment (fish, amphibians, plants, insects, etc.) have an influence on these phenomena. Research has focused on the synthesis of analogous compounds that are sufficiently stable and whose activity is at least equal to or even greater than the activity of the natural molecules, for commercial applications.

Anti-freeze proteins (AFP) have generated increasing interest for their ability to protect cells under a variety of conditions. They are naturally encountered in Arctic and Antarctic fish as well as other cold-climate dwelling invertebrates and are responsible for maintaining cells and tissue functional at sub-zero temperatures. AFP were successfully isolated in the 1950s and have demonstrated the ability to non-colligatively lower the freezing temperature of body fluids by binding to ice crystals.

Early experiments with these compounds in the field of organ and tissue transplantation showed promising results, making them attractive therapeutic candidates to protect cells against harmful conditions associated with the process of retrieval-preservation-reperfusion. Moreover, further benefits have also been demonstrated during cryopreservation of different cells, including islets of Langerhans, which significantly increased their viability and function when supplemented with AFP during cryostasis. The Anti-aging glycopeptides (AAGP™) used in the present invention derive from attempts to obtain analogs of anti-freeze glycoproteins.

Anti-aging glycopeptides (AAGP™) compounds are gem difluorinated C-glycopeptides which have been proposed to have applicability under harsh cellular stresses, such as nutrient deprivation, high temperature and cryopreservation, oxidative stress from hydrogen peroxide (H₂O₂), UV irradiation, and inflammation.

Cell, organ (or portions thereof), and tissue transplantation is a promising approach to replace tissue lost because of diseases. However, it has become evident that the survival and functional integration rate of transplanted cells, organs or tissues over the long term remains an issue, in part because of immune rejection disease leading to immune rejection, and also because of the toxicity of immunosuppressant drugs leading to loss of functionality of the graft.

Therefore, there exists a need in the art for means of improving graft survival.

### SUMMARY

According to an embodiment, there is provided a method for inhibition or prevention of immune rejection of a graft comprising the step of:
a) contacting an isolated graft prior to a transplantation in a subject in need thereof, with a gem-difluorinated C-glycopeptide compound of general formula I, or a pharmaceutically acceptable base, addition salt with an acid, hydrate or solvate of the compound of general formula I:

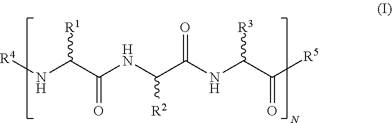

in which:
N is an integer between 1 and 5,
R⁴═H, AA₁, or AA₁-AA₂,
R⁵═OH, AA₁, or AA₁-AA₂,
AA₁ and AA₂ independently represent amino acids with a non-polar side chain and
R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

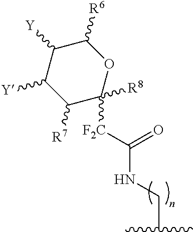

in which:
n is an integer between 3 and 4,
Y, Y′ are independent groups
   in which Y, Y′ ═H, OR, N₃, NR′R″, or SR′″, where R═H, benzyl, trimethylsilyl, tert-butyldimethylsilyl, tert-butyldiphenylsilyl or acetate group,
   R′, R″ independently=H, alkyl, allyl, benzyl, tosylate group, C(═O)-alkyl or C(═O)—Bn,
   R′″═H, alkyl, or acetate group,
R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
R³ is a hydrogen atom H or a free or protected alcohol function, and
   if R¹═R²═H, CH₃, CH₂Ph, CH(CH₃)₂, CH₂CH(CH₃)₂, or CH(CH₃)CH₂CH₃
   then R³=

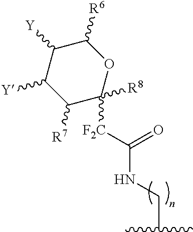

in which: n is an integer between 3 and 4,
Y, Y′ are independent groups
   in which Y, Y′ ═H, OR, N₃, NR′R″, or SR′,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tertbutyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R¹═R³═H, CH₃, CH₂Ph, CH(CH₃)₂, CH₂CH(CH₃)₂, or CH(CH₃)CH₂CH₃
   then R²=

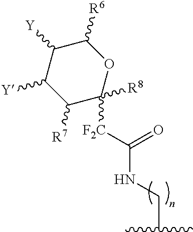

in which: n is an integer between 3 and 4,
Y, Y′ are independent groups
   in which Y, Y′ ═H, OR, N₃, NR′R″, or SR′,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R²═R³═H, CH₃, CH(CH₃)₂, CH₂CH(CH₃)₂, or CH(CH₃)CH₂CH₃
   then R¹=

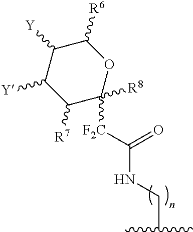

in which: n is an integer between 3 and 4,
Y, Y′ are independent groups
   in which Y, Y′ H, OR, N₃, NR′R″, or SR′″,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn, R′″═H, alkyl, or acetate group,
   R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
   R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
   R³ is a hydrogen atom H or a free or protected alcohol function
      b) transplanting the graft in the subject in need thereof, the subject in need thereof being under an immune suppressant drug therapy to inhibit or prevent immune rejection of the graft;
      c) discontinue the immune suppressant drug therapy after a time sufficient for implantation of the graft in the subject in need thereof.
      The method may further comprise step a′) before step a), a′) isolating the graft.

The immunosuppressant drug may be one of sirolimus, tacrolimus, cyclosporine, everolimus or combinations thereof.

The subject may be a human subject.

The isolated graft may be isolated from a live donor, a cadaveric donor, or combinations thereof.

The compound of formula I may be a compound of formula II:

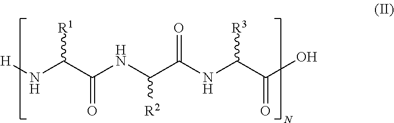

in which: N is an integer between 1 and 5, and
R¹, R², R³ are independent groups in which two of R¹, R² and R³ are selected from H, CH₃ and the remaining R¹, R² and R³ is

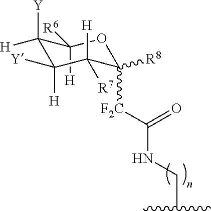

in which: n is an integer between 3 and 4,
Y, Y′ are independent groups
   in which Y, Y′ ═H, OR, N₃, NR′R″ or SR′″,
      where R═H, benzyl, trimethylsilyl, tert-butyldimethylsilyl, tert-butyldiphenylsilyl, or acetate group,
      R′, R″ independently=H, alkyl, allyl, benzyl, tosylate group, C(═O)-alkyl, or C(═O)—Bn,
      R′″═H, alkyl, or acetate group,
R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl or acetate group,
R⁸ is a hydrogen atom H or a free or protected alcohol function,
   and
   if R¹═R²═H or CH₃,
   then R³=

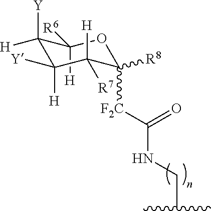

in which: n is an integer between 3 and 4,
Y, Y′ are independent groups
   in which Y, Y′ ═H, OR, N₃, NR′R″, or SR′″,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″═H, alkyl, or acetate group,
R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   if R¹═R³═H or CH₃,
   then R²=

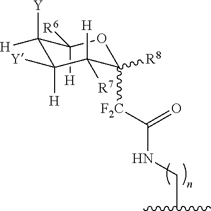

in which: n is an integer between 3 and 4,
Y, Y′ are independent groups
   in which Y, Y′ ═H, OR, N₃, NR′R″, SR′″,
   where R═H, benzyl, trimethylsilyl, tert-butyldimethylsilyl, tert-butyldiphenylsilyl, or acetate group,
   R′, R″ independently=H, alkyl, allyl, Bn, tosylate, C(═O)-alkyl, or C(═O)—Bn,
   R′″═H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   if R²═R³═H or CH₃,
   then R¹=

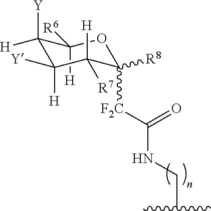

in which: n is an integer between 3 and 4,
Y, Y′ are independent groups
   in which Y, Y′H, OR, N₃, NR′R″, or SR′″, where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn, R′═H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷═OH, OGP, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
   R⁸ is a hydrogen atom or a free or protected alcohol function.

The compound of formula I may be a compound of formula II:

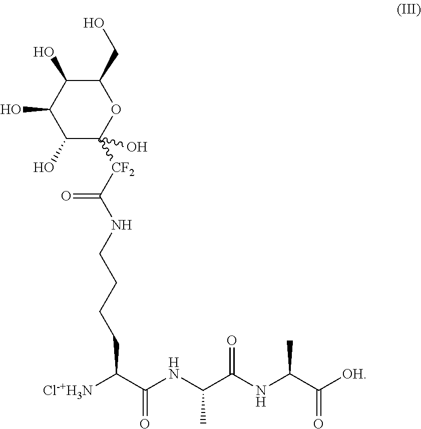

The contacting of the isolated graft may be with from about from about 0.01 mg/ml to about 5 mg/ml of the compound of formula I, formula II or formula III.

The contacting of the isolated graft may be with from about from about 1 mg/ml to about 5 mg/ml of the compound of formula I, formula II or formula III.

The isolated graft comprises an organ, a fragment of an organ, a tissue, an isolated cell, or combinations thereof.

The organ or the fragment of an organ may be a liver, a heart, a kidney, a lung, a pancreas, an intestine, a thymus, a uterus, a stomach, a testis, a penis, a hand.

The tissue may be a bone, a bone marrow, a tendon, a cornea, a heart valve, a skin, and a blood vessel.

The isolated cell may be any one of an isolated pancreatic cell, and isolated pancreatic progenitor cell, an adult stem cells, a neurosensory precursor cell, a reticular cell, a glial cell, a neural cell, a cardiac myocyte, an hepatocyte, and an hematopoietic stem cell.

The isolated pancreatic cell may be an isolated alpha cell, an isolated beta cell, an isolated delta cell, an isolated gamma cell, an epsilon cell, or a combination thereof.

The isolated pancreatic cell may be an isolated beta cell.

The neurosensory precursor cell may be a photoreceptor precursor cell.

The graft may be contacted with the compound for about 1 minute to about 1 hour prior to transplantation.

In the method of the present invention, the organ may be a pancreas, and the of may be for the treatment of diabetes.

In the method of the present invention, the isolated cell may be a neurosensory precursor cell, and the method may be for treating a retinal degenerative disease.

The retinal degenerative disease may be age-related macular degeneration (AMD), retinitis pigmentosa (RP), retinal vasculitis, or sarcoidosis.

The isolated graft may be washed to remove the compound of formula I, II or Ill.

According to another embodiment, there may be provided a use of a gem-difluorinated C-glycopeptide compound of general formula I, or a pharmaceutically acceptable base, addition salt with an acid, hydrate or solvate of the compound of general formula I for inhibition or prevention of immune rejection of an isolated graft contacted with the compound, prior to transplantation in a subject in need thereof:

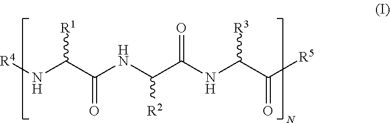

in which:
N is an integer between 1 and 5,
R⁴═H, AA₁, or AA₁-AA₂,
R⁵═OH, AA₁, or AA₁-AA₂,
AA₁ and AA₂ independently represent amino acids with a non-polar side chain and
R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

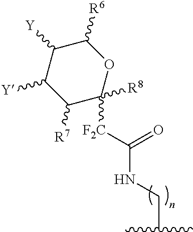

in which:
n is an integer between 3 and 4,
Y, Y′ are independent groups
   in which Y, Y′ ═H, OR, N₃, NR′R″, or SR′″,
      where R═H, benzyl, trimethylsilyl, tert-butyldimethylsilyl, tert-butyldiphenylsilyl or acetate group,
      R′, R″ independently=H, alkyl, allyl, benzyl, tosylate group, C(═O)-alkyl or C(═O)—Bn,
      R′″═H, alkyl, or acetate group,
R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function, and
   if R¹═R²═H, CH₃, CH₂Ph, CH(CH₃)₂, CH₂CH(CH₃)₂, or CH(CH₃)CH₂CH₃
   then R³=

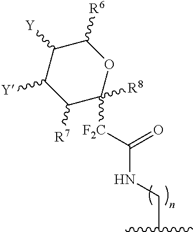

in which: n is an integer between 3 and 4,
Y, Y′ are independent groups
   in which Y, Y′ ═H, OR, N₃, NR′R″, or SR′,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tertbutyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R¹═R³═H, CH₃, CH₂Ph, CH(CH₃)₂, CH₂CH(CH₃)₂, or CH(CH₃)CH₂CH₃
   then R²=

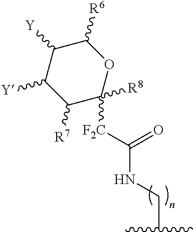

in which: n is an integer between 3 and 4,
Y, Y′ are independent groups
   in which Y, Y′ ═H, OR, N₃, NR′R″, or SR′,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R²═R³═H, CH₃, CH(CH₃)₂, CH₂CH(CH₃)₂, or CH(CH₃)CH₂CH₃
   then R¹=

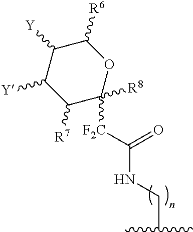

in which: n is an integer between 3 and 4,
Y, Y′ are independent groups
   in which Y, Y′ H, OR, N₃, NR′R″, or SR′″,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function.

The subject in need thereof may be under an immune suppressant drug therapy to inhibit or prevent immune rejection of the graft.

The immune suppressant drug therapy may be discontinued after a time sufficient for implantation of the graft in the subject in need thereof.

The immunosuppressant drug may be one of sirolimus, tacrolimus, cyclosporine, everolimus or combinations thereof.

The subject may be a human subject.

The isolated graft may be isolated from a live donor, a cadaveric donor, or combinations thereof.

The compound of formula I may be a compound of formula II:

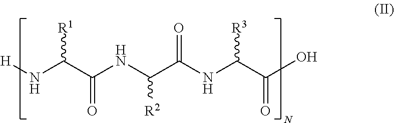

in which: N is an integer between 1 and 5, and
R¹, R², R³ are independent groups in which two of R¹, R² and R³ are selected from H, CH₃ and the remaining R¹, R² and R³ is

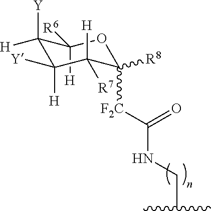

in which: n is an integer between 3 and 4,
Y, Y′ are independent groups
   in which Y, Y′ ═H, OR, N₃, NR′R″ or SR′″,
      where R═H, benzyl, trimethylsilyl, tert-butyldimethylsilyl, tert-butyldiphenylsilyl, or acetate group,
      R′, R″ independently=H, alkyl, allyl, benzyl, tosylate group, C(═O)-alkyl, or C(═O)—Bn,
      R′″═H, alkyl, or acetate group,
R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl or acetate group,
R⁸ is a hydrogen atom H or a free or protected alcohol function,
   and
   if R¹═R²═H or CH₃,
   then R³=

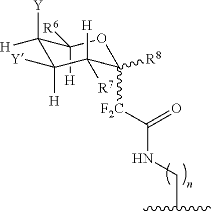

in which: n is an integer between 3 and 4,
Y, Y′ are independent groups
   in which Y, Y′ ═H, OR, N₃, NR′R″, or SR′″,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″═H, alkyl, or acetate group,
R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   if R¹═R³═H or CH₃,
   then R²=

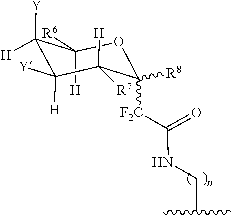

in which: n is an integer between 3 and 4,
Y, Y′ are independent groups
   in which Y, Y′ ═H, OR, N₃, NR′R″, SR′″,
   where R═H, benzyl, trimethylsilyl, tert-butyldimethylsilyl, tert-butyldiphenylsilyl, or acetate group,
   R′, R″ independently=H, alkyl, allyl, Bn, tosylate, C(═O)-alkyl, or C(═O)—Bn,
   R′″═H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   if R²═R³═H or CH₃,
   then R¹=

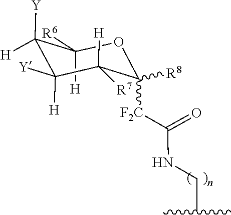

in which: n is an integer between 3 and 4,
Y, Y′ are independent groups
   in which Y, Y′ H, OR, N₃, NR′R″, or SR′″,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″═H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom or a free or protected alcohol function.

The compound of formula I may be a compound of formula III:

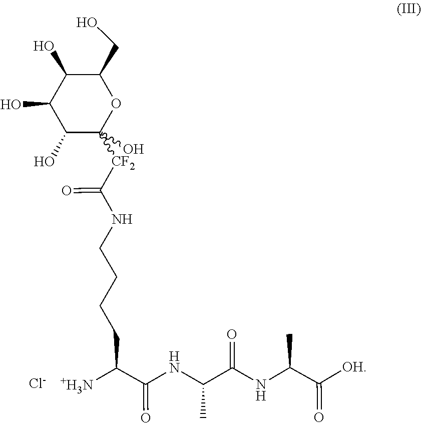

The contacting of the isolated graft may be with from about from about 0.01 mg/ml to about 5 mg/ml of the compound of formula I, formula II or formula III.

The contacting of the isolated graft may be with from about from about 1 mg/ml to about 5 mg/ml of the compound of formula I, formula II or formula III.

The isolated graft comprises an organ, a fragment of an organ, a tissue, an isolated cell, or combinations thereof.

The organ or the fragment of an organ may be a liver, a heart, a kidney, a lung, a pancreas, an intestine, a thymus, a uterus, a stomach, a testis, a penis, a hand.

The tissue may be a bone, a bone marrow, a tendon, a cornea, a heart valve, a skin, and a blood vessel.

The isolated cell may be any one of an isolated pancreatic cell, and isolated pancreatic progenitor cell, an adult stem cells, a neurosensory precursor cell, a reticular cell, a glial cell, a neural cell, a cardiac myocyte, an hepatocyte, and an hematopoietic stem cell.

The isolated pancreatic cell may be an isolated alpha cell, an isolated beta cell, an isolated delta cell, an isolated gamma cell, an epsilon cell, or a combination thereof.

The isolated pancreatic cell may be an isolated beta cell.

The neurosensory precursor cell may be a photoreceptor precursor cell.

The isolated graft may be contacted with the compound for about 1 minute to about 1 hour prior to transplantation.

The isolated graft may be washed to remove the compound of formula I, II or Ill.

In the use of the present invention, the organ may be a pancreas, and the use may be for the treatment of diabetes.

In the use of the present invention, the isolated cell may be a neurosensory precursor cell, and the use may be for treating a retinal degenerative disease.

The retinal degenerative disease may be age-related macular degeneration (AMD), retinitis pigmentosa (RP), retinal vasculitis, or sarcoidosis.

According to another embodiment, there is provided a method for inhibition or prevention of immune rejection comprising the step of:
a) contacting an isolated graft prior to a transplantation in a subject in need thereof, with a gem-difluorinated C-glycopeptide compound of general formula I, or a pharmaceutically acceptable base, addition salt with an acid, hydrate or solvate of the compound of general formula I

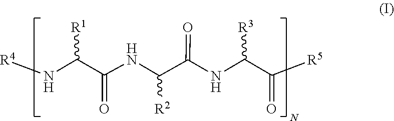

in which:
N is an integer between 1 and 5,
   R⁴═H, AA₁, or AA₁-AA₂,
   R⁵═OH, AA₁, or AA₁-AA₂,
   AA₁ and AA₂ independently represent amino acids with a non-polar side chain and
   R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

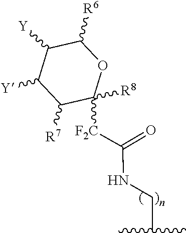

in which:
n is an integer between 3 and 4,
   Y, Y′ are independent groups
      in which Y, Y′ ═H, OR, N₃, NR′R″, or SR′″,
      where R═H, benzyl, trimethylsilyl, tert-butyldimethylsilyl, tert-butyldiphenylsilyl or acetate group,
      R′, R″ independently=H, alkyl, allyl, benzyl, tosylate group, C(═O)-alkyl or C(═O)—Bn,
      R′″═H, alkyl, or acetate group,
      R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
      R⁷═OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
      R⁸ is a hydrogen atom H or a free or protected alcohol function, and
      if R¹═R²═H, CH₃, CH₂Ph, CH(CH₃)₂, CH₂CH(CH₃)₂, or CH(CH₃)CH₂CH₃
      then R³=

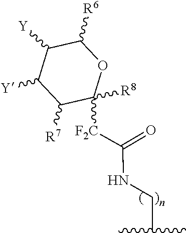

in which: n is an integer between 3 and 4,
Y, Y′ are independent groups
in which Y, Y′ ═H, OR, N₃, NR′R″, or SR′,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn, R′″═H, alkyl, or acetate group,
   R⁶ is H, CH₃, CH₂OH, CH₂-glycoside group, or CH₂-OGP in which GP is a protector group selected from alkyl, benzyl, trimethylsilyl, tertbutyldimethylsilyl, tert-butyldiphenylsilyl, or acetate group,
   R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
   R⁸ is a hydrogen atom H or a free or protected alcohol function,
   if R¹═R³═H, CH₃, CH₂Ph, CH(CH₃)₂, CH₂CH(CH₃)₂, or CH(CH₃)CH₂CH₃
   then R²=

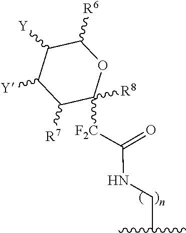

in which: n is an integer between 3 and 4,
Y, Y′ are independent groups
in which Y, Y′ ═H, OR, N₃, NR′R″, or SR′,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn, R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
if R²═R³═H, CH₃, CH(CH₃)₂, CH₂CH(CH₃)₂, or CH(CH₃)CH₂CH₃
then R¹=

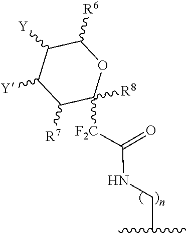

in which: n is an integer between 3 and 4,
Y, Y′ are independent groups
in which Y, Y′ H, OR, N₃, NR′R″, or SR′″,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn, R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl,
tert-butyldiphenylsilyl, or acetate group,
R⁸ is a hydrogen atom H or a free or protected alcohol function,
b) transplanting the graft in the subject in need thereof, the subject in need thereof receiving no immune suppressant drug therapy to inhibit or prevent immune rejection against the graft.

According to another embodiment, there is provided a gem-difluorinated C-glycopeptide compound of general formula I, or a pharmaceutically acceptable base, addition salt with an acid, hydrate or solvate of the compound of general formula I for use in inhibition or prevention of immune rejection of an isolated graft contacted with the compound, prior to transplantation in a subject in need thereof:

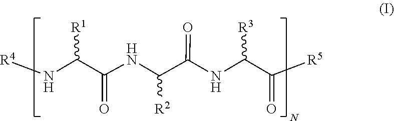

in which:
N is an integer between 1 and 5,
R⁴═H, AA₁, or AA₁-AA₂,
R⁵═OH, AA₁, or AA₁-AA₂,
AA₁ and AA₂ independently represent amino acids with a non-polar side chain and
R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

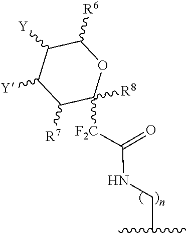

in which:
   n is an integer between 3 and 4,
   Y, Y′ are independent groups
      in which Y, Y′ ═H, OR, N₃, NR′R″, or SR′″,
         where R═H, benzyl, trimethylsilyl, tert-butyldimethylsilyl, tert-butyldiphenylsilyl or acetate group,
         R′, R″ independently=H, alkyl, allyl, benzyl, tosylate group, C(═O)-alkyl or C(═O)—Bn,
         R′″═H, alkyl, or acetate group,
   R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
   R⁷═OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
   R⁸ is a hydrogen atom H or a free or protected alcohol function, and
      if R¹═R²═H, CH₃, CH₂Ph, CH(CH₃)₂, CH₂CH(CH₃)₂, or CH(CH₃)CH₂CH₃
      then R³=

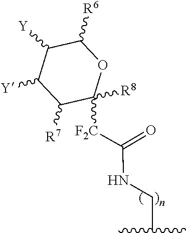

in which: n is an integer between 3 and 4,
Y, Y′ are independent groups
   in which Y, Y′ ═H, OR, N₃, NR′R″, or SR′,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tertbutyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R¹═R³═H, CH₃, CH₂Ph, CH(CH₃)₂, CH₂CH(CH₃)₂, or CH(CH₃)CH₂CH₃
   then R²=

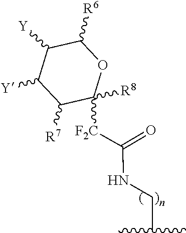

in which: n is an integer between 3 and 4,
Y, Y′ are independent groups
   in which Y, Y′ ═H, OR, N₃, NR′R″, or SR′,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R²═R³═H, CH₃, CH(CH₃)₂, CH₂CH(CH₃)₂, or CH(CH₃)CH₂CH₃
   then R¹=

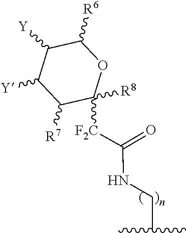

in which: n is an integer between 3 and 4,
Y, Y′ are independent groups
   in which Y, Y′ H, OR, N₃, NR′R″, or SR′″,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function.

The subject in need thereof may be under an immune suppressant drug therapy to inhibit or prevent immune rejection of the graft.

The immune suppressant drug therapy may be discontinued after a time sufficient for implantation of the graft in the subject in need thereof.

According to another embodiment, there is provided an isolated graft contacted with a gem-difluorinated C-glycopeptide compound of general formula I, or a pharmaceutically acceptable base, addition salt with an acid, hydrate or solvate of the compound of general formula I prior to transplantation in a subject in need thereof, for inhibition or prevention of immune rejection of the isolated graft contacted with the compound:

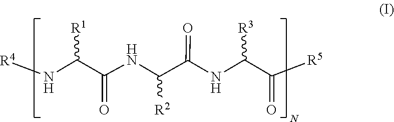

in which:
N is an integer between 1 and 5,
R⁴═H, AA₁, or AA₁-AA₂,
R⁵═OH, AA₁, or AA₁-AA₂,
AA₁ and AA₂ independently represent amino acids with a non-polar side chain and
R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

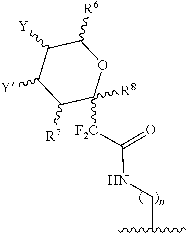

in which:
n is an integer between 3 and 4,
Y, Y′ are independent groups
   in which Y, Y′ ═H, OR, N₃, NR′R″, or SR′″,
      where R═H, benzyl, trimethylsilyl, tert-butyldimethylsilyl, tert-butyldiphenylsilyl or acetate group,
      R′, R″ independently=H, alkyl, allyl, benzyl, tosylate group, C(═O)-alkyl or C(═O)—Bn,
      R′″═H, alkyl, or acetate group,
   R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
   R⁷═OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
   R⁸ is a hydrogen atom H or a free or protected alcohol function, and
      if R¹═R²═H, CH₃, CH₂Ph, CH(CH₃)₂, CH₂CH(CH₃)₂, or CH(CH₃)CH₂CH₃
      then R³=

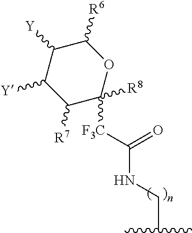

in which: n is an integer between 3 and 4,
Y, Y′ are independent groups
   in which Y, Y′ ═H, OR, N₃, NR′R″, or SR′,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tertbutyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R¹═R³═H, CH₃, CH₂Ph, CH(CH₃)₂, CH₂CH(CH₃)₂, or CH(CH₃)CH₂CH₃
   then R²=

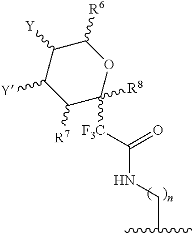

in which: n is an integer between 3 and 4,
Y, Y′ are independent groups
   in which Y, Y′ ═H, OR, N₃, NR′R″, or SR′,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R²═R³═H, CH₃, CH(CH₃)₂, CH₂CH(CH₃)₂, or CH(CH₃)CH₂CH₃
   then R¹=

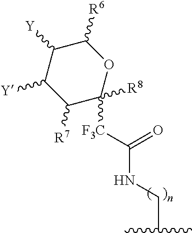

in which: n is an integer between 3 and 4,
Y, Y′ are independent groups
   in which Y, Y′ H, OR, N₃, NR′R″, or SR′″,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function.

The subject in need thereof may be under an immune suppressant drug therapy to inhibit or prevent immune rejection of the graft.

The immune suppressant drug therapy may be discontinued after a time sufficient for implantation of the graft in the subject in need thereof.

The following terms are defined below.

The term «immunosuppression» as used herein is intended to mean the reduction of the activation or efficacy of the immune system. Some portions of the immune system itself have immunosuppressive effects on other parts of the immune system, and immunosuppression may occur as an adverse reaction to treatment of other conditions. But in the context of the present invention more specifically, immunosuppression is performed to prevent the body from rejecting an organ transplant. through immune rejection disease. A person who is undergoing immunosuppression, or whose immune system is weak for some other reasons is the to be immunocompromised.

The term «discontinue» as used herein regarding the discontinuation of the immune suppressant drug therapy in intended to mean to cease providing the immune suppressant drug. In the context of the present invention, this is after a time sufficient for implantation of the transplanted graft, as defined below.

The term «graft» as used herein is intended to mean a piece of living organ, portion(s) thereof, tissues and/or cells that is transplanted surgically. Types of grafts generically encompass autograft which is a graft taken from one part of the body of an individual and transplanted onto another site in the same individual, e.g., skin graft; isograft which is a graft taken from one individual and placed on another individual of the same genetic constitution, e.g., grafts between identical twins; allograft: graft taken from one individual placed on genetically non-identical member of the same species and xenograft, which is a graft taken from one individual placed on an individual belonging to another species, e.g., animal to man. In the context of the present invention, a graft usually refers to allografts and xenografts that are subject to immune rejection. According to an embodiment, the graft may be one that has been cryopreserved by freezing for example in liquid nitrogen. According to another distinct embodiment, the graft may be one which has not been subjected to any cryopreservation and/or freezing of any form. Therefore, the method or uses of the present invention would comprises no step where the isolated graft is cryopreserved.

The terms «graft implantation» and «implantation» as used herein is intended to mean the insertion, fixing or attachment of the graft into a person's body.

The term «time sufficient for implantation» as used herein is intended to mean the time it takes for a transplanted graft to attach and/or get fixed into the person's body. This time will be variable and depend upon the type of graft being transplanted. It may range from a few minutes, a few hours, a day or a few days, a week or a few weeks to months. The time sufficient may be from about 1 minute to about 10 minutes, or from about 1 minute to about 15 minutes, or from about 1 minute to about 30 minutes, or from about 1 minute to about 45 minutes, or from about 1 minute to about 1 hour, or from about 1 minute to about 2 hours, or from about 1 minute to about 6 hours, or from about 1 minute to about 12 hours, or from about 1 minute to about 18 hours, or from about 1 minute to about 1 day, or from about 1 minute to about 2 days, or from about 1 minute to about 3 days, or from about 1 minute to about 4 days, or from about 1 minute to about 5 days, or from about 1 minute to about 6 days, or from about 1 minute to about 1 week, or from about 1 minute to about 2 weeks, or from about 1 minute to about 3 weeks, or from about 1 minute to about 1 month, or from about 1 minute to about 2 months, or from about 1 minute to about 3 months, or from about 1 minute to about 4 months, or from about 1 minute to about 5 months, or from about 1 minute to about 6 months, or from about 10 minutes to about 15 minutes, or from about 10 minutes to about 30 minutes, or from about 10 minutes to about 45 minutes, or from about 10 minutes to about 1 hour, or from about 10 minutes to about 2 hours, or from about 10 minutes to about 6 hours, or from about 10 minutes to about 12 hours, or from about 10 minutes to about 18 hours, or from about 10 minutes to about 1 day, or from about 10 minutes to about 2 days, or from about 10 minutes to about 3 days, or from about 10 minutes to about 4 days, or from about 10 minutes to about 5 days, or from about 10 minutes to about 6 days, or from about 10 minutes to about 1 week, or from about 10 minutes to about 2 weeks, or from about 10 minutes to about 3 weeks, or from about 10 minutes to about 1 month, or from about 10 minutes to about 2 months, or from about 10 minutes to about 3 months, or from about 10 minutes to about 4 months, or from about 10 minutes to about 5 months, or from about 10 minutes to about 6 months, or from about 15 minutes to about 30 minutes, or from about 15 minutes to about 45 minutes, or from about 15 minutes to about 1 hour, or from about 15 minutes to about 2 hours, or from about 15 minutes to about 6 hours, or from about 15 minutes to about 12 hours, or from about 15 minutes to about 18 hours, or from about 15 minutes to about 1 day, or from about 15 minutes to about 2 days, or from about 15 minutes to about 3 days, or from about 15 minutes to about 4 days, or from about 15 minutes to about 5 days, or from about 15 minutes to about 6 days, or from about 15 minutes to about 1 week, or from about 15 minutes to about 2 weeks, or from about 15 minutes to about 3 weeks, or from about 15 minutes to about 1 month, or from about 15 minutes to about 2 months, or from about 15 minutes to about 3 months, or from about 15 minutes to about 4 months, or from about 15 minutes to about 5 months, or from about 15 minutes to about 6 months, or from about 30 minutes to about 45 minutes, or from about 30 minutes to about 1 hour, or from about 30 minutes to about 2 hours, or from about 30 minutes to about 6 hours, or from about 30 minutes to about 12 hours, or from about 30 minutes to about 18 hours, or from about 30 minutes to about 1 day, or from about 30 minutes to about 2 days, or from about 30 minutes to about 3 days, or from about 30 minutes to about 4 days, or from about 30 minutes to about 5 days, or from about 30 minutes to about 6 days, or from about 30 minutes to about 1 week, or from about 30 minutes to about 2 weeks, or from about 30 minutes to about 3 weeks, or from about 30 minutes to about 1 month, or from about 30 minutes to about 2 months, or from about 30 minutes to about 3 months, or from about 30 minutes to about 4 months, or from about 30 minutes to about 5 months, or from about 30 minutes to about 6 months, or from about 45 minutes to about 1 hour, or from about 45 minutes to about 2 hours, or from about 45 minutes to about 6 hours, or from about 45 minutes to about 12 hours, or from about 45 minutes to about 18 hours, or from about 45 minutes to about 1 day, or from about 45 minutes to about 2 days, or from about 45 minutes to about 3 days, or from about 45 minutes to about 4 days, or from about 45 minutes to about 5 days, or from about 45 minutes to about 6 days, or from about 45 minutes to about 1 week, or from about 45 minutes to about 2 weeks, or from about 45 minutes to about 3 weeks, or from about 45 minutes to about 1 month, or from about 45 minutes to about 2 months, or from about 45 minutes to about 3 months, or from about 45 minutes to about 4 months, or from about 45 minutes to about 5 months, or from about 45 minutes to about 6 months, or from about 1 hour to about 2 hours, or from about 1 hour to about 6 hours, or from about 1 hour to about 12 hours, or from about 1 hour to about 18 hours, or from about 1 hour to about 1 day, or from about 1 hour to about 2 days, or from about 1 hour to about 3 days, or from about 1 hour to about 4 days, or from about 1 hour to about 5 days, or from about 1 hour to about 6 days, or from about 1 hour to about 1 week, or from about 1 hour to about 2 weeks, or from about 1 hour to about 3 weeks, or from about 1 hour to about 1 month, or from about 1 hour to about 2 months, or from about 1 hour to about 3 months, or from about 1 hour to about 4 months, or from about 1 hour to about 5 months, or from about 1 hour to about 6 months, or from about 2 hours to about 6 hours, or from about 2 hours to about 12 hours, or from about 2 hours to about 18 hours, or from about 2 hours to about 1 day, or from about 2 hours to about 2 days, or from about 2 hours to about 3 days, or from about 2 hours to about 4 days, or from about 2 hours to about 5 days, or from about 2 hours to about 6 days, or from about 2 hours to about 1 week, or from about 2 hours to about 2 weeks, or from about 2 hours to about 3 weeks, or from about 2 hours to about 1 month, or from about 2 hours to about 2 months, or from about 2 hours to about 3 months, or from about 2 hours to about 4 months, or from about 2 hours to about 5 months, or from about 2 hours to about 6 months, or from about 6 hours to about 12 hours, or from about 6 hours to about 18 hours, or from about 6 hours to about 1 day, or from about 6 hours to about 2 days, or from about 6 hours to about 3 days, or from about 6 hours to about 4 days, or from about 6 hours to about 5 days, or from about 6 hours to about 6 days, or from about 6 hours to about 1 week, or from about 6 hours to about 2 weeks, or from about 6 hours to about 3 weeks, or from about 6 hours to about 1 month, or from about 6 hours to about 2 months, or from about 6 hours to about 3 months, or from about 6 hours to about 4 months, or from about 6 hours to about 5 months, or from about 6 hours to about 6 months, or from about 12 hours to about 18 hours, or from about 12 hours to about 1 day, or from about 12 hours to about 2 days, or from about 12 hours to about 3 days, or from about 12 hours to about 4 days, or from about 12 hours to about 5 days, or from about 12 hours to about 6 days, or from about 12 hours to about 1 week, or from about 12 hours to about 2 weeks, or from about 12 hours to about 3 weeks, or from about 12 hours to about 1 month, or from about 12 hours to about 2 months, or from about 12 hours to about 3 months, or from about 12 hours to about 4 months, or from about 12 hours to about 5 months, or from about 12 hours to about 6 months, or from about 18 hours to about 1 day, or from about 18 hours to about 2 days, or from about 18 hours to about 3 days, or from about 18 hours to about 4 days, or from about 18 hours to about 5 days, or from about 18 hours to about 6 days, or from about 18 hours to about 1 week, or from about 18 hours to about 2 weeks, or from about 18 hours to about 3 weeks, or from about 18 hours to about 1 month, or from about 18 hours to about 2 months, or from about 18 hours to about 3 months, or from about 18 hours to about 4 months, or from about 18 hours to about 5 months, or from about 18 hours to about 6 months, or from about 1 day to about 2 days, or from about 1 day to about 3 days, or from about 1 day to about 4 days, or from about 1 day to about 5 days, or from about 1 day to about 6 days, or from about 1 day to about 1 week, or from about 1 day to about 2 weeks, or from about 1 day to about 3 weeks, or from about 1 day to about 1 month, or from about 1 day to about 2 months, or from about 1 day to about 3 months, or from about 1 day to about 4 months, or from about 1 day to about 5 months, or from about 1 day to about 6 months, or from about 2 days to about 3 days, or from about 2 days to about 4 days, or from about 2 days to about 5 days, or from about 2 days to about 6 days, or from about 2 days to about 1 week, or from about 2 days to about 2 weeks, or from about 2 days to about 3 weeks, or from about 2 days to about 1 month, or from about 2 days to about 2 months, or from about 2 days to about 3 months, or from about 2 days to about 4 months, or from about 2 days to about 5 months, or from about 2 days to about 6 months, or from about 3 days to about 4 days, or from about 3 days to about 5 days, or from about 3 days to about 6 days, or from about 3 days to about 1 week, or from about 3 days to about 2 weeks, or from about 3 days to about 3 weeks, or from about 3 days to about 1 month, or from about 3 days to about 2 months, or from about 3 days to about 3 months, or from about 3 days to about 4 months, or from about 3 days to about 5 months, or from about 3 days to about 6 months, or from about 4 days to about 5 days, or from about 4 days to about 6 days, or from about 4 days to about 1 week, or from about 4 days to about 2 weeks, or from about 4 days to about 3 weeks, or from about 4 days to about 1 month, or from about 4 days to about 2 months, or from about 4 days to about 3 months, or from about 4 days to about 4 months, or from about 4 days to about 5 months, or from about 4 days to about 6 months, or from about 5 days to about 6 days, or from about 5 days to about 1 week, or from about 5 days to about 2 weeks, or from about 5 days to about 3 weeks, or from about 5 days to about 1 month, or from about 5 days to about 2 months, or from about 5 days to about 3 months, or from about 5 days to about 4 months, or from about 5 days to about 5 months, or from about 5 days to about 6 months, or from about 6 days to about 1 week, or from about 6 days to about 2 weeks, or from about 6 days to about 3 weeks, or from about 6 days to about 1 month, or from about 6 days to about 2 months, or from about 6 days to about 3 months, or from about 6 days to about 4 months, or from about 6 days to about 5 months, or from about 6 days to about 6 months, or from about 1 week to about 2 weeks, or from about 1 week to about 3 weeks, or from about 1 week to about 1 month, or from about 1 week to about 2 months, or from about 1 week to about 3 months, or from about 1 week to about 4 months, or from about 1 week to about 5 months, or from about 1 week to about 6 months, or from about 2 weeks to about 3 weeks, or from about 2 weeks to about 1 month, or from about 2 weeks to about 2 months, or from about 2 weeks to about 3 months, or from about 2 weeks to about 4 months, or from about 2 weeks to about 5 months, or from about 2 weeks to about 6 months, or from about 3 weeks to about 1 month, or from about 3 weeks to about 2 months, or from about 3 weeks to about 3 months, or from about 3 weeks to about 4 months, or from about 3 weeks to about 5 months, or from about 3 weeks to about 6 months, or from about 1 month to about 2 months, or from about 1 month to about 3 months, or from about 1 month to about 4 months, or from about 1 month to about 5 months, or from about 1 month to about 6 months, or from about 2 months to about 3 months, or from about 2 months to about 4 months, or from about 2 months to about 5 months, or from about 2 months to about 6 months, or from about 3 months to about 4 months, or from about 3 months to about 5 months, or from about 3 months to about 6 months, or from about 4 months to about 5 months, or from about 4 months to about 6 months, or from about 5 months to about 6 months.

The terms «host-versus-graft disease» and «graft-versus-host disease» as used herein refers to a syndrome characterized by inflammation in different organs commonly associated with any type of transplant in a subject having received a transplant, including solid organ transplants, parts of solid organ transplants, cell transplants such as stem cell transplants such as those that occur with bone marrow transplants, pancreatic cells transplants, where either the graft, or the host elicits an immune response against the host or graft, respectively.

The terms «transplant» or «organ transplantation» as used herein a medical procedure in which an organ is removed from one body and placed in the body of a recipient, to replace a damaged or missing organ, portion(s) thereof, tissues and/or cells. The donor and recipient may be at the same location, or organs may be transported from a donor site to another location. Organs, portion(s) thereof, tissues and/or cells that are transplanted within the same person's body are called autografts. Transplants that are recently performed between two subjects of the same species are called allografts. Allografts can either be from a living or cadaveric source.

The terms “inhibit”, “inhibition” or “inhibiting” as used herein in the context of the invention means to slow, hinder, restrain, reduce, delay, protect against or prevent. For example, “inhibiting host-versus-graft disease” or, “inhibiting graft-versus-host disease” or “inhibiting immune rejection” of organs, portion(s) thereof, tissues and/or cells as that term is used herein means to slow, hinder, restrain, reduce, reduce, protect against or prevent host-versus-graft/graft-versus-host disease and/or immune rejection and/or the onset of immune rejection.

The term «immune rejection» or «transplant rejection» as used herein refers to when a transplanted organ, portion(s) thereof, tissues and/or cells are rejected by the recipient's immune system, which destroys the transplanted organs, portion(s) thereof, tissues and/or cells. Transplant rejection can be lessened by determining the molecular similitude between donor and recipient and by use of immunosuppressant drugs after transplant.

The term «contacting» as used herein is intended to mean touching the organs, portion(s) thereof, tissues and/or cells with the compound of the present invention, for a sufficient amount of time to provide the effect(s) imparted by the compounds. In some embodiments, contacting is intended to mean incubating the organs, portion(s) thereof, tissues and/or cells with a suitable solution comprising the compound. In some embodiments, this may comprise perfusion of the organs, portion(s) thereof, tissues (i.e. the passage of blood, a blood substitute, or other fluid through the blood vessels or other natural channels in an organ or tissue).

The term «organ» as used herein is intended to mean a part of an organism that is typically self-contained and has a specific vital function, such as the heart or liver in humans.

The term a «subject» is preferably a human subject but can also be any mammal, including an animal model. Mammals of interest include, but are not limited to: rodents, e.g. mice, rats; livestock, e.g. pigs, horses, cows, etc., pets, e.g. dogs, cats; and primates. A subject may also be referred to herein as a “patient”.

The term «composition» as used herein is intended to encompass a product comprising the specified ingredients in the specified amounts, as well as any product which results, directly or indirectly, from combination of the specified ingredients in the specified amounts. Such term in relation to pharmaceutical composition or other compositions in general, is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions or other compositions in general of the present invention encompass any composition made by admixing a compound of the present invention and a pharmaceutically acceptable carrier. By “pharmaceutically acceptable” or “acceptable” it is meant the carrier, diluent or excipient must be compatible with the other ingredients of the formulation and not deleterious to the recipient thereof.

The terms “neurosensory precursor cell”, are intended to mean cells derived from human embryonic stem cells or induced pluripotent stem cells, isolated through known protocols and/or as described herein below, as well as cells of grown in vivo, ex vivo and/or in vitro.

The terms “pancreatic cell”, “isolated pancreatic cell”, “islet cells”, or “isolated islet cells” are intended to mean cells derived from the Islet of Langerhans from the pancreas from live or cadaveric donors isolated through known protocols and/or as described herein below, as well as cells of pancreatic origin grown in vivo, ex vivo and/or in vitro. The cells of the Islet of Langerhans include alpha cells, producing the hormone glucagon and representing about 15-20% of the islet cells, the beta cells producing the hormones insulin and amylin, and representing about 65-80% of the islet cells, the delta cells producing the hormone somatostatin and representing about 3-10% of the islet cells, the PP cells (also known as gamma cells) producing the hormone pancreatic polypeptide (3-5% of the islet cells, and the epsilon cells producing the hormone ghrelin, representing <1% of the islet cells.

The terms “pancreatic beta cell” and “isolated pancreatic beta cell” is intended to mean cells derived from pancreas from live or cadaveric donors isolated through known protocols and/or as described herein below, as well as beta cells of pancreatic origin grown in vivo, ex vivo and/or in vitro.

The terms “pancreatic progenitor” and “isolated pancreatic progenitor” is intended to mean cells derived from any suitable sources, such as embryonic stem cells (of human or other origin), that have differentiated or are differentiate naturally or through known protocols into pancreatic progenitor cells in vivo, ex vivo or in vitro. The isolated pancreatic progenitor cells have the potential to become pancreatic beta cells through further differentiation naturally or through treatment with known protocols, in vivo, ex vivo or in vitro. For example, the isolated pancreatic progenitor cells may be obtained in vitro through a differentiation protocol, and further differentiated into pancreatic beta cells in vitro through a differentiation protocol. Also, the isolated pancreatic progenitor cells may be obtained in vitro through a differentiation protocol, and further differentiated into pancreatic beta cells in vivo after implantation/transplantation into a patient in need thereof.

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. Where the specified number of carbon atoms permits, e.g., from C3-10, the term alkyl also includes cycloalkyl groups, and combinations of linear or branched alkyl chains combined with cycloalkyl structures. When no number of carbon atoms is specified, C1-6 is intended.

“Cycloalkyl” is a subset of alkyl and means a saturated carbocyclic ring having a specified number of carbon atoms. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

The term “alkoxy” refers to straight or branched chain alkoxides of the number of carbon atoms specified (e.g., C1-6 alkoxy), or any number within this range [i.e., methoxy (MeO−), ethoxy, isopropoxy, etc.].

The term “alkylthio” refers to straight or branched chain alkylsulfides of the number of carbon atoms specified (e.g., C1-6 alkylthio), or any number within this range [i.e., methylthio (MeS−), ethylthio, isopropylthio, etc.].

The term “alkylamino” refers to straight or branched alkylamines of the number of carbon atoms specified (e.g., C1-6 alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C1-6 alkylsulfonyl), or any number within this range [i.e., methylsulfonyl (MeSO₂⁻), ethylsulfonyl, isopropylsulfonyl, etc.].

The term “alkylsulfinyl” refers to straight or branched chain alkylsulfoxides of the number of carbon atoms specified (e.g., C1-6 alkylsulfinyl), or any number within this range [i.e., methylsulfinyl (MeSO−), ethylsulfinyl, isopropylsulfinyl, etc.].

The term “alkyloxycarbonyl” refers to straight or branched chain esters of a carboxylic acid derivative of the present invention of the number of carbon atoms specified (e.g., C1-6 alkyloxycarbonyl), or any number within this range [i.e., methyloxycarbonyl (MeOCO⁻), ethyloxycarbonyl, or butyloxycarbonyl].

“Aryl” means a mono- or polycyclic aromatic ring system containing carbon ring atoms. The preferred aryls are monocyclic or bicyclic 6-10 membered aromatic ring systems. Phenyl and naphthyl are preferred aryls. The most preferred aryl is phenyl.

“Heterocyclyl” refer to saturated or unsaturated non-aromatic rings or ring systems containing at least one heteroatom selected from O, S and N, further including the oxidized forms of sulfur, namely SO and SO₂. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, 2-oxopiperidin-1-yl, 2-oxopyrrolidin-1-yl, 2-oxoazetidin-1-yl, 1,2,4-oxadiazin-5(6H)-one-3-yl, and the like.

“Heteroaryl” means an aromatic or partially aromatic heterocycle that contains at least one ring heteroatom selected from O, S and N. Heteroaryls thus include heteroaryls fused to other kinds of rings, such as aryls, cycloalkyls and heterocycles that are not aromatic. Examples of heteroaryl groups include: pyrrolyl, isoxazolyl, isothiazolyl, pyrazolyl, pyridyl, oxazolyl, oxadiazolyl (in particular, 1,3,4-oxadiazol-2-yl and 1,2,4-oxadiazol-3-yl), thiadiazolyl, thiazolyl, imidazolyl, triazolyl, tetrazolyl, furyl, triazinyl, thienyl, pyrimidyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl, and the like. For heterocyclyl and heteroaryl groups, rings and ring systems containing from 3-15 atoms are included, forming 1-3 rings.

“Halogen” refers to fluorine, chlorine, bromine and iodine. Chlorine and fluorine are generally preferred. Fluorine is most preferred when the halogens are substituted on an alkyl or alkoxy group (e.g. CF₃O and CF₃CH₂O).

Before describing the present invention in detail, a number of terms will be defined. As used herein, the singular forms “a”, “an”, and “the” include plural referents unless the context clearly dictates otherwise.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 is a flow chart of the study design presented in Example 1.

FIG. 2 is a flow chart of the procedure used to generate Photoreceptor precursor cells (PPCs) from pluripotent human ES cells (hESC) used in Example 1.

FIG. 3 is a flowchart of rabbit treatment and tissue processing for Example 1.

FIG. 4 illustrates flatmount images from control (PPC cells without AAGP™) and treatment (PPC cells with AAGP™) groups at 6 months post transplantation.

FIG. 5A illustrates the CD4+ T cells in the conjunctival epithelium after acute dry eye disease induction.

FIG. 5B illustrates the CD4+ T cells in the conjunctival epithelium after acute dry eye disease induction.

It will be noted that throughout the appended drawings, like features are identified by like reference numerals.

### DETAILED DESCRIPTION

In embodiments there is disclosed a method for inhibition or prevention of immune rejection comprising the step of:

a) contacting an isolated graft prior to a transplantation in a subject in need thereof, with a gem-difluorinated C-glycopeptide compound of general formula I, or a pharmaceutically acceptable base, addition salt with an acid, hydrate or solvate of the compound of general formula I

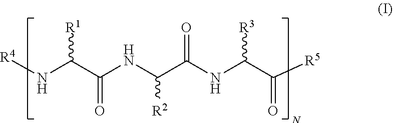

in which:
N is an integer between 1 and 5,
   R⁴═H, AA₁, or AA₁-AA₂,
   R⁵═OH, AA₁, or AA₁-AA₂,
   AA₁ and AA₂ independently represent amino acids with a non-polar side chain and
   R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

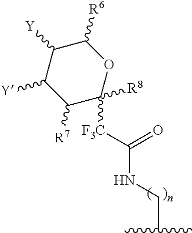

in which:
n is an integer between 3 and 4,
   Y, Y′ are independent groups
      in which Y, Y′ ═H, OR, N₃, NR′R″, or SR′″,
      where R═H, benzyl, trimethylsilyl, tert-butyldimethylsilyl, tert-butyldiphenylsilyl or acetate group,
      R′, R″ independently=H, alkyl, allyl, benzyl, tosylate group, C(═O)-alkyl or C(═O)—Bn,
      R′″═H, alkyl, or acetate group,
      R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
      R⁷═OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
      R⁸ is a hydrogen atom H or a free or protected alcohol function, and
      if R¹═R²═H, CH₃, CH₂Ph, CH(CH₃)₂, CH₂CH(CH₃)₂, or CH(CH₃)CH₂CH₃
      then R³=

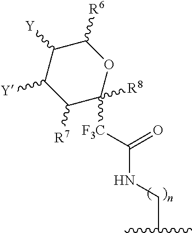

in which: n is an integer between 3 and 4,
Y, Y′ are independent groups
in which Y, Y′ ═H, OR, N₃, NR′R″, or SR′,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″═H, alkyl, or acetate group,
   R⁶ is H, CH₃, CH₂OH, CH₂-glycoside group, or CH₂-OGP in which GP is a protector group selected from alkyl, benzyl, trimethylsilyl, tertbutyldimethylsilyl, tert-butyldiphenylsilyl, or acetate group,
   R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
   R⁸ is a hydrogen atom H or a free or protected alcohol function,
   if R¹═R³═H, CH₃, CH₂Ph, CH(CH₃)₂, CH₂CH(CH₃)₂, or CH(CH₃)CH₂CH₃
   then R²=

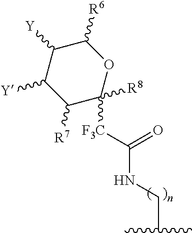

in which: n is an integer between 3 and 4,
Y, Y′ are independent groups
in which Y, Y′ ═H, OR, N₃, NR′R″, or SR′,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
if R²═R³═H, CH₃, CH(CH₃)₂, CH₂CH(CH₃)₂, or CH(CH₃)CH₂CH₃
then R¹=

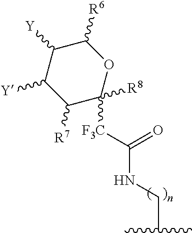

in which: n is an integer between 3 and 4,
Y, Y′ are independent groups
in which Y, Y′ H, OR, N₃, NR′R″, or SR′″,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function
   b) transplanting the graft in the subject in need thereof, the subject in need thereof being under an immune suppressant drug therapy to inhibit or prevent immune rejection against the graft;
   c) discontinue the immune suppressant drug therapy after a time sufficient for implantation of the graft in the subject in need thereof.

The method may further comprise step a′) before step a), a′) isolating the graft.

The immunosuppressant drug may be one of sirolimus, tacrolimus, cyclosporine, everolimus or combinations thereof.

The subject may be a human subject.

The subject may be under an immune suppressant drug therapy to inhibit or prevent immune rejection against the graft for at least about 1 hour, 2 hours, 6 hours, 12 hours, 1 day, 2 days, 3 days, 4 days, 5 days, 6 days, 1 week, 2 weeks, 3 weeks or 4 weeks, or for about 1 hour to about 2 hours, or for about 1 hour to about 6 hours, or for about 1 hour to about 12 hours, or for about 1 hour to about 1 day, or for about 1 hour to about 2 days, or for about 1 hour to about 3 days, or for about 1 hour to about 4 days, or for about 1 hour to about 5 days, or for about 1 hour to about 6 days, or for about 1 hour to about 1 week, or for about 1 hour to about 2 weeks, or for about 1 hour to about 3 weeks, or for about 1 hour to about 4 weeks, or for about 2 hours to about 6 hours, or for about 2 hours to about 12 hours, or for about 2 hours to about 1 day, or for about 2 hours to about 2 days, or for about 2 hours to about 3 days, or for about 2 hours to about 4 days, or for about 2 hours to about 5 days, or for about 2 hours to about 6 days, or for about 2 hours to about 1 week, or for about 2 hours to about 2 weeks, or for about 2 hours to about 3 weeks, or for about 2 hours to about 4 weeks, or for about 3 hours to about 12 hours, or for about 3 hours to about 1 day, or for about 3 hours to about 2 days, or for about 3 hours to about 3 days, or for about 3 hours to about 4 days, or for about 3 hours to about 5 days, or for about 3 hours to about 6 days, or for about 3 hours to about 1 week, or for about 3 hours to about 2 weeks, or for about 3 hours to about 3 weeks, or for about 3 hours to about 4 weeks, or for about 6 hours to about 12 hours, or for about 6 hours to about 1 day, or for about 6 hours to about 2 days, or for about 6 hours to about 3 days, or for about 6 hours to about 4 days, or for about 6 hours to about 5 days, or for about 6 hours to about 6 days, or for about 6 hours to about 1 week, or for about 6 hours to about 2 weeks, or for about 6 hours to about 3 weeks, or for about 6 hours to about 4 weeks, or for about 12 hours to about 1 day, or for about 12 hours to about 2 days, or for about 12 hours to about 3 days, or for about 12 hours to about 4 days, or for about 12 hours to about 5 days, or for about 12 hours to about 6 days, or for about 12 hours to about 1 week, or for about 12 hours to about 2 weeks, or for about 12 hours to about 3 weeks, or for about 12 hours to about 4 weeks, or for about 1 day to about 2 days, or for about 1 day to about 3 days, or for about 1 day to about 4 days, or for about 1 day to about 5 days, or for about 1 day to about 6 days, or for about 1 day to about 1 week, or for about 1 day to about 2 weeks, or for about 1 day to about 3 weeks, or for about 1 day to about 4 weeks, or for about 2 days to about 3 days, or for about 2 days to about 4 days, or for about 2 days to about 5 days, or for about 2 days to about 6 days, or for about 2 days to about 1 week, or for about 2 days to about 2 weeks, or for about 2 days to about 3 weeks, or for about 2 days to about 4 weeks, or for about 3 days to about 4 days, or for about 3 days to about 5 days, or for about 3 days to about 6 days, or for about 3 days to about 1 week, or for about 3 days to about 2 weeks, or for about 3 days to about 3 weeks, or for about 3 days to about 4 weeks, or for about 4 days to about 5 days, or for about 4 days to about 6 days, or for about 4 days to about 1 week, or for about 4 days to about 2 weeks, or for about 4 days to about 3 weeks, or for about 4 days to about 4 weeks, or for about 5 days to about 6 days, or for about 5 days to about 1 week, or for about 5 days to about 2 weeks, or for about 5 days to about 3 weeks, or for about 5 days to about 4 weeks, or for about 6 days to about 1 week, or for about 6 days to about 2 weeks, or for about 6 days to about 3 weeks, or for about 6 days to about 4 weeks, or for about 1 week to about 2 weeks, or for about 1 week to about 3 weeks, or for about 1 week to about 4 weeks, or for about 2 weeks to about 3 weeks, or for about 2 weeks to about 4 weeks, or for about 3 weeks to about 4 weeks prior to transplantation of the graft.

The isolated graft may be isolated from a live donor, a cadaveric donor, or combinations thereof.

The compound of formula I may be a compound of formula II:

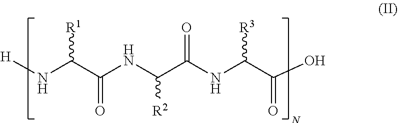

in which: N is an integer between 1 and 5, and
R¹, R², R³ are independent groups in which two of R¹, R² and R³ are selected from H, CH₃ and the
remaining R¹, R² and R³ is

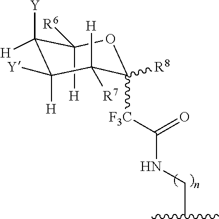

in which: n is an integer between 3 and 4,
Y, Y′ are independent groups
   in which Y, Y′ ═H, OR, N₃, NR′R″ or SR′″,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″═H, alkyl, or acetate group,
   R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
   R⁷═OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl or acetate group,
   R⁸ is a hydrogen atom H or a free or protected alcohol function, and
   if R¹═R²═H or CH₃,
   then R³=

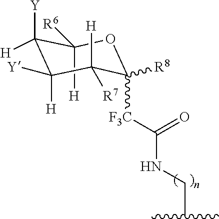

in which: n is an integer between 3 and 4,
Y, Y′ are independent groups
in which Y, Y′ ═H, OR, N₃, NR′R″, or SR′‘ ’,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″═H, alkyl, or acetate group,
R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
if R¹═R³═H or CH₃,
then R²=

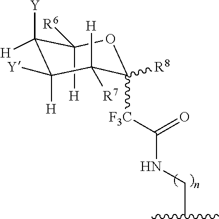

in which: n is an integer between 3 and 4,
Y, Y′ are independent groups
in which Y, Y′═H, OR, N₃, NR′R″, SR′″,
where R═H, benzyl, trimethylsilyl, tert-butyldimethylsilyl, tert-butyldiphenylsilyl, or acetate group,
R′, R″ independently=H, alkyl, allyl,
Bn, tosylate, C(═O)-alkyl, or C(═O)—Bn,
R′″═H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
if R²═R³═H or CH₃,
then R¹=

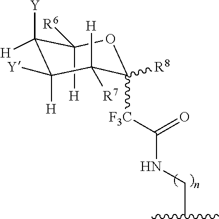

in which: n is an integer between 3 and 4,
Y, Y′ are independent groups
in which Y, Y′ H, OR, N₃, NR′R″, or SR′″,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″═H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom or a free or protected alcohol function.

The compound of formula I may be a compound of formula III:

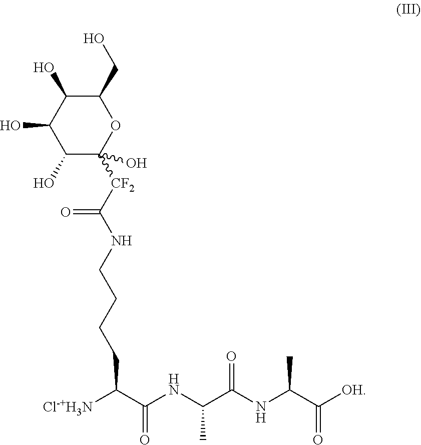

Contacting the isolated graft may be with from about from about 0.01 mg/ml to about 5 mg/ml of the compound of formula I, formula II or formula III. Contacting the isolated graft may be with from about from about 1 mg/ml to about 5 mg/ml of the compound of formula I, formula II or formula III.

The isolated graft may comprise an organ, a fragment of an organ, a tissue, an isolated cell, or combinations thereof. The organ or the fragment of an organ may be a liver, a heart, a kidney, a lung, a pancreas, an intestine, a thymus, a uterus, a stomach, a testis, a penis, a hand. The tissue may be a bone, a bone marrow, a tendon, a cornea, a heart valve, a skin, and a blood vessel. The wherein said isolated cell is any one of an isolated pancreatic cell, and isolated pancreatic progenitor cell, an adult stem cells, a neurosensory precursor cell, a reticular cell, a glial cell, a neural cell, a cardiac myocyte, an hepatocyte, and an hematopoietic stem cell. The isolated pancreatic cell may be an isolated alpha cell, an isolated beta cell, an isolated delta cell, an isolated gamma cell, an epsilon cell, or a combination thereof. The isolated pancreatic cell may be an isolated beta cell. The neurosensory precursor cell may be a photoreceptor precursor cell. According to an embodiment, the graft may be one that has been cryopreserved by freezing for example in liquid nitrogen. According to another distinct embodiment, the graft may be one which has not been subjected to any cryopreservation and/or freezing of any form. Therefore, the method or uses of the present invention would comprises no step where the isolated graft is cryopreserved.

The graft may be contacted with the compound for at least about 1 minute, or about 15 minutes, or about 30 minutes, or about 1 hour, or 6 hours, or about 12 hours, or about 24 hours, or about 48 hours, or about 72 hours, or about 1 minute to about 15 minutes, or for about 1 minute to about 30 minutes, or for about 1 minute to about 1 hour, or for about 1 minute to about 6 hours, or for about 1 minute to about 12 hours, or for about 1 minute to about 24 hours, or for about 1 minute to about 48 hours, or for about 1 minute to about 72 hours, or for about 15 minutes to about 30 minutes, or for about 15 minutes to about 1 hour, or for about 15 minutes to about 6 hours, or for about 15 minutes to about 12 hours, or for about 15 minutes to about 24 hours, or for about 15 minutes to about 48 hours, or for about 15 minutes to about 72 hours, or for about 30 minutes to about 1 hour, or for about 30 minutes to about 6 hours, or for about 30 minutes to about 12 hours, or for about 30 minutes to about 24 hours, or for about 30 minutes to about 48 hours, or for about 30 minutes to about 72 hours, or for about 1 hour to about 6 hours, or for about 1 hour to about 12 hours, or for about 1 hour to about 24 hours, or for about 1 hour to about 48 hours, or for about 1 hour to about 72 hours, or for about 2 hours to about 12 hours, or for about 2 hours to about 24 hours, or for about 2 hours to about 48 hours, or for about 2 hours to about 72 hours, or for about 6 hours to about 12 hours, or for about 6 hours to about 24 hours, or for about 6 hours to about 48 hours, or for about 6 hours to about 72 hours, or for about 12 hours to about 24 hours, or for about 12 hours to about 48 hours, or for about 12 hours to about 72 hours, or for about 24 hours to about 48 hours, or for about 24 hours to about 72 hours, or for about 24 hours to about 72 hours, or for 1 min, 15 mins, 30 mins, 1 hour, 2 hours, 6 hours, 12 hours, 24 hours, 48 hours, or 72 hours prior to transplantation.

The period after which the immune suppressant drug therapy is discontinued, or in other words the time sufficient for implantation may be from at least about 1 hour, 2 hours, 6 hours, 12 hours, 1 day, 2 days, 3 days, 4 days, 5 days, 6 days, 1 week, 2 weeks, 3 weeks or 4 weeks, or for about 1 hour to about 2 hours, or for about 1 hour to about 6 hours, or for about 1 hour to about 12 hours, or for about 1 hour to about 1 day, or for about 1 hour to about 2 days, or for about 1 hour to about 3 days, or for about 1 hour to about 4 days, or for about 1 hour to about 5 days, or for about 1 hour to about 6 days, or for about 1 hour to about 1 week, or for about 1 hour to about 2 weeks, or for about 1 hour to about 3 weeks, or for about 1 hour to about 4 weeks, or for about 2 hours to about 6 hours, or for about 2 hours to about 12 hours, or for about 2 hours to about 1 day, or for about 2 hours to about 2 days, or for about 2 hours to about 3 days, or for about 2 hours to about 4 days, or for about 2 hours to about 5 days, or for about 2 hours to about 6 days, or for about 2 hours to about 1 week, or for about 2 hours to about 2 weeks, or for about 2 hours to about 3 weeks, or for about 2 hours to about 4 weeks, or for about 3 hours to about 12 hours, or for about 3 hours to about 1 day, or for about 3 hours to about 2 days, or for about 3 hours to about 3 days, or for about 3 hours to about 4 days, or for about 3 hours to about 5 days, or for about 3 hours to about 6 days, or for about 3 hours to about 1 week, or for about 3 hours to about 2 weeks, or for about 3 hours to about 3 weeks, or for about 3 hours to about 4 weeks, or for about 6 hours to about 12 hours, or for about 6 hours to about 1 day, or for about 6 hours to about 2 days, or for about 6 hours to about 3 days, or for about 6 hours to about 4 days, or for about 6 hours to about 5 days, or for about 6 hours to about 6 days, or for about 6 hours to about 1 week, or for about 6 hours to about 2 weeks, or for about 6 hours to about 3 weeks, or for about 6 hours to about 4 weeks, or for about 12 hours to about 1 day, or for about 12 hours to about 2 days, or for about 12 hours to about 3 days, or for about 12 hours to about 4 days, or for about 12 hours to about 5 days, or for about 12 hours to about 6 days, or for about 12 hours to about 1 week, or for about 12 hours to about 2 weeks, or for about 12 hours to about 3 weeks, or for about 12 hours to about 4 weeks, or for about 1 day to about 2 days, or for about 1 day to about 3 days, or for about 1 day to about 4 days, or for about 1 day to about 5 days, or for about 1 day to about 6 days, or for about 1 day to about 1 week, or for about 1 day to about 2 weeks, or for about 1 day to about 3 weeks, or for about 1 day to about 4 weeks, or for about 2 days to about 3 days, or for about 2 days to about 4 days, or for about 2 days to about 5 days, or for about 2 days to about 6 days, or for about 2 days to about 1 week, or for about 2 days to about 2 weeks, or for about 2 days to about 3 weeks, or for about 2 days to about 4 weeks, or for about 3 days to about 4 days, or for about 3 days to about 5 days, or for about 3 days to about 6 days, or for about 3 days to about 1 week, or for about 3 days to about 2 weeks, or for about 3 days to about 3 weeks, or for about 3 days to about 4 weeks, or for about 4 days to about 5 days, or for about 4 days to about 6 days, or for about 4 days to about 1 week, or for about 4 days to about 2 weeks, or for about 4 days to about 3 weeks, or for about 4 days to about 4 weeks, or for about 5 days to about 6 days, or for about 5 days to about 1 week, or for about 5 days to about 2 weeks, or for about 5 days to about 3 weeks, or for about 5 days to about 4 weeks, or for about 6 days to about 1 week, or for about 6 days to about 2 weeks, or for about 6 days to about 3 weeks, or for about 6 days to about 4 weeks, or for about 1 week to about 2 weeks, or for about 1 week to about 3 weeks, or for about 1 week to about 4 weeks, or for about 2 weeks to about 3 weeks, or for about 2 weeks to about 4 weeks, or for about 3 weeks to about 4 weeks after transplantation of the graft.

In the method of the present invention, the organ may be a pancreas, and the method may be for the treatment of diabetes.

In the method of the present invention, the isolated cell may be a neurosensory precursor cell, and the method may be for treating a retinal degenerative disease. The retinal degenerative disease may be age-related macular degeneration (AMD), retinitis pigmentosa (RP), retinal vasculitis, or sarcoidosis.

In embodiments. the isolated graft may be washed to remove the compound of formula I, II or Ill, prior to a transplantation in the subject in need thereof.

In embodiments there is disclosed a method for inhibition or prevention of immune rejection comprising the step of:
a) contacting an isolated graft prior to a transplantation in a subject in need thereof, with a gem-difluorinated C-glycopeptide compound of general formula I, or a pharmaceutically acceptable base, addition salt with an acid, hydrate or solvate of the compound of general formula I

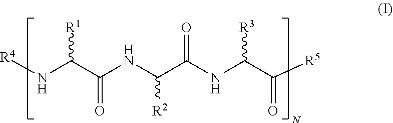

in which:
N is an integer between 1 and 5,
   R⁴═H, AA₁, or AA₁-AA₂,
   R⁵═OH, AA₁, or AA₁-AA₂,
   AA₁ and AA₂ independently represent amino acids with a non-polar side chain and
   R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

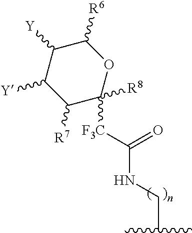

in which:
n is an integer between 3 and 4,
   Y, Y′ are independent groups
      in which Y, Y′ ═H, OR, N₃, NR′R″, or SR′″,
      where R═H, benzyl, trimethylsilyl, tert-butyldimethylsilyl, tert-butyldiphenylsilyl or acetate group,
      R′, R″ independently=H, alkyl, allyl, benzyl, tosylate group, C(═O)-alkyl or C(═O)—Bn,
      R′″═H, alkyl, or acetate group,
      R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
      R⁷═OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
      R⁸ is a hydrogen atom H or a free or protected alcohol function, and
      if R¹═R²═H, CH₃, CH₂Ph, CH(CH₃)₂, CH₂CH(CH₃)₂, or CH(CH₃)CH₂CH₃
      then R³=

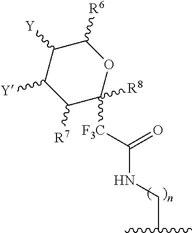

in which: n is an integer between 3 and 4,
Y, Y′ are independent groups
in which Y, Y′ ═H, OR, N₃, NR′R″, or SR′,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″═H, alkyl, or acetate group,
   R⁶ is H, CH₃, CH₂OH, CH₂-glycoside group, or CH₂-OGP in which GP is a protector group selected from alkyl, benzyl, trimethylsilyl, tertbutyldimethylsilyl, tert-butyldiphenylsilyl, or acetate group,
   R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
   R⁸ is a hydrogen atom H or a free or protected alcohol function,
   if R¹═R³═H, CH₃, CH₂Ph, CH(CH₃)₂, CH₂CH(CH₃)₂, or CH(CH₃)CH₂CH₃
   then R²=

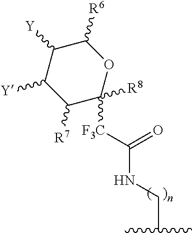

in which: n is an integer between 3 and 4,
Y, Y′ are independent groups
in which Y, Y′ ═H, OR, N₃, NR′R″, or SR′,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
if R²═R³═H, CH₃, CH(CH₃)₂, CH₂CH(CH₃)₂, or CH(CH₃)CH₂CH₃
then R¹=

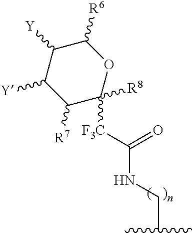

in which: n is an integer between 3 and 4,
Y, Y′ are independent groups
in which Y, Y′ H, OR, N₃, NR′R″, or SR′″,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl,
tert-butyldiphenylsilyl, or acetate group,
R⁸ is a hydrogen atom H or a free or protected alcohol function,
b) transplanting the graft in the subject in need thereof, the subject in need thereof receiving no immune suppressant drug therapy to inhibit or prevent immune rejection against the graft.

For example, the subject in need thereof receiving no immune suppressant drug therapy may be an immunocompromised subject, or a subject having a weak immune system. Examples of persons with weakened immune systems according to this embodiment include those with HIV/AIDS; cancer; and those with inherited diseases that affect the immune system (e.g., congenital agammaglobulinemia, congenital IgA deficiency). The risk of developing severe disease may differ depending on each person's degree of immune suppression.

According to another embodiment, there is provided a use of a gem-difluorinated C-glycopeptide compound of general formula I, or a pharmaceutically acceptable base, addition salt with an acid, hydrate or solvate of the compound of general formula I for inhibition or prevention of immune rejection against an isolated graft contacted with the compound, prior to transplantation in a subject in need thereof:

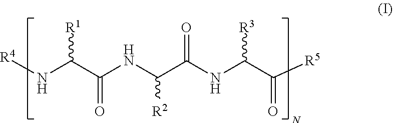

in which:
N is an integer between 1 and 5,
   R⁴═H, AA₁, or AA₁-AA₂,
   R⁵═OH, AA₁, or AA₁-AA₂,
   AA₁ and AA₂ independently represent amino acids with a non-polar side chain and
   R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

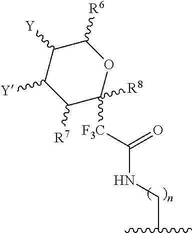

in which:
n is an integer between 3 and 4,
   Y, Y′ are independent groups
      in which Y, Y′ ═H, OR, N₃, NR′R″, or SR′″,
      where R═H, benzyl, trimethylsilyl, tert-butyldimethylsilyl, tert-butyldiphenylsilyl or acetate group,
      R′, R″ independently=H, alkyl, allyl, benzyl, tosylate group, C(═O)-alkyl or C(═O)—Bn,
      R′″═H, alkyl, or acetate group,
      R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
      R⁷═OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
      R⁸ is a hydrogen atom H or a free or protected alcohol function, and
      if R¹═R²═H, CH₃, CH₂Ph, CH(CH₃)₂, CH₂CH(CH₃)₂, or CH(CH₃)CH₂CH₃
      then R³=

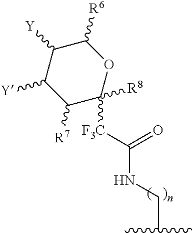

in which: n is an integer between 3 and 4,
Y, Y′ are independent groups
in which Y, Y′ ═H, OR, N₃, NR′R″, or SR′,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″═H, alkyl, or acetate group,
   R⁶ is H, CH₃, CH₂OH, CH₂-glycoside group, or CH₂-OGP in which GP is a protector group selected from alkyl, benzyl, trimethylsilyl, tertbutyldimethylsilyl, tert-butyldiphenylsilyl, or acetate group,
   R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
   R⁸ is a hydrogen atom H or a free or protected alcohol function,
   if R¹═R³═H, CH₃, CH₂Ph, CH(CH₃)₂, CH₂CH(CH₃)₂, or CH(CH₃)CH₂CH₃
   then R²=

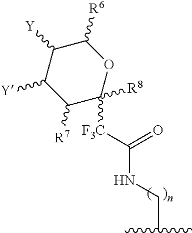

in which: n is an integer between 3 and 4,
Y, Y′ are independent groups
in which Y, Y′ ═H, OR, N₃, NR′R″, or SR′,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
if R²═R³═H, CH₃, CH(CH₃)₂, CH₂CH(CH₃)₂, or CH(CH₃)CH₂CH₃
then R¹=

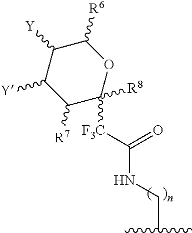

in which: n is an integer between 3 and 4,
Y, Y′ are independent groups
in which Y, Y′ H, OR, N₃, NR′R″, or SR′″,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl,
tert-butyldiphenylsilyl, or acetate group,
R⁸ is a hydrogen atom H or a free or protected alcohol function.

In use, the compound may be used for about 1 minute to about 15 minutes, or for about 1 minute to about 30 minutes, or for about 1 minute to about 1 hour, or for about 1 minute to about 6 hours, or for about 1 minute to about 12 hours, or for about 1 minute to about 24 hours, or for about 1 minute to about 48 hours, or for about 1 minute to about 72 hours, or for about 15 minutes to about 30 minutes, or for about 15 minutes to about 1 hour, or for about 15 minutes to about 6 hours, or for about 15 minutes to about 12 hours, or for about 15 minutes to about 24 hours, or for about 15 minutes to about 48 hours, or for about 15 minutes to about 72 hours, or for about 30 minutes to about 1 hour, or for about 30 minutes to about 6 hours, or for about 30 minutes to about 12 hours, or for about 30 minutes to about 24 hours, or for about 30 minutes to about 48 hours, or for about 30 minutes to about 72 hours, or for about 1 hour to about 6 hours, or for about 1 hour to about 12 hours, or for about 1 hour to about 24 hours, or for about 1 hour to about 48 hours, or for about 1 hour to about 72 hours, or for about 2 hours to about 12 hours, or for about 2 hours to about 24 hours, or for about 2 hours to about 48 hours, or for about 2 hours to about 72 hours, or for about 6 hours to about 12 hours, or for about 6 hours to about 24 hours, or for about 6 hours to about 48 hours, or for about 6 hours to about 72 hours, or for about 12 hours to about 24 hours, or for about 12 hours to about 48 hours, or for about 12 hours to about 72 hours, or for about 24 hours to about 48 hours, or for about 24 hours to about 72 hours, or for about 24 hours to about 72 hours, or for 1 min, 15 mins, 30 mins, 1 hour, 2 hours, 6 hours, 12 hours, 24 hours, 48 hours, or 72 hours prior to transplantation.

In the use of the present invention, the subject in need thereof is under an immune suppressant drug therapy to inhibit or prevent immune rejection against the graft. The subject may be under under an immune suppressant drug therapy to inhibit or prevent immune rejection against the graft for at least about 1 hour, 2 hours, 6 hours, 12 hours, 1 day, 2 days, 3 days, 4 days, 5 days, 6 days, 1 week, 2 weeks, 3 weeks or 4 weeks, or for about 1 hour to about 2 hours, or for about 1 hour to about 6 hours, or for about 1 hour to about 12 hours, or for about 1 hour to about 1 day, or for about 1 hour to about 2 days, or for about 1 hour to about 3 days, or for about 1 hour to about 4 days, or for about 1 hour to about 5 days, or for about 1 hour to about 6 days, or for about 1 hour to about 1 week, or for about 1 hour to about 2 weeks, or for about 1 hour to about 3 weeks, or for about 1 hour to about 4 weeks, or for about 2 hours to about 6 hours, or for about 2 hours to about 12 hours, or for about 2 hours to about 1 day, or for about 2 hours to about 2 days, or for about 2 hours to about 3 days, or for about 2 hours to about 4 days, or for about 2 hours to about 5 days, or for about 2 hours to about 6 days, or for about 2 hours to about 1 week, or for about 2 hours to about 2 weeks, or for about 2 hours to about 3 weeks, or for about 2 hours to about 4 weeks, or for about 3 hours to about 12 hours, or for about 3 hours to about 1 day, or for about 3 hours to about 2 days, or for about 3 hours to about 3 days, or for about 3 hours to about 4 days, or for about 3 hours to about 5 days, or for about 3 hours to about 6 days, or for about 3 hours to about 1 week, or for about 3 hours to about 2 weeks, or for about 3 hours to about 3 weeks, or for about 3 hours to about 4 weeks, or for about 6 hours to about 12 hours, or for about 6 hours to about 1 day, or for about 6 hours to about 2 days, or for about 6 hours to about 3 days, or for about 6 hours to about 4 days, or for about 6 hours to about 5 days, or for about 6 hours to about 6 days, or for about 6 hours to about 1 week, or for about 6 hours to about 2 weeks, or for about 6 hours to about 3 weeks, or for about 6 hours to about 4 weeks, or for about 12 hours to about 1 day, or for about 12 hours to about 2 days, or for about 12 hours to about 3 days, or for about 12 hours to about 4 days, or for about 12 hours to about 5 days, or for about 12 hours to about 6 days, or for about 12 hours to about 1 week, or for about 12 hours to about 2 weeks, or for about 12 hours to about 3 weeks, or for about 12 hours to about 4 weeks, or for about 1 day to about 2 days, or for about 1 day to about 3 days, or for about 1 day to about 4 days, or for about 1 day to about 5 days, or for about 1 day to about 6 days, or for about 1 day to about 1 week, or for about 1 day to about 2 weeks, or for about 1 day to about 3 weeks, or for about 1 day to about 4 weeks, or for about 2 days to about 3 days, or for about 2 days to about 4 days, or for about 2 days to about 5 days, or for about 2 days to about 6 days, or for about 2 days to about 1 week, or for about 2 days to about 2 weeks, or for about 2 days to about 3 weeks, or for about 2 days to about 4 weeks, or for about 3 days to about 4 days, or for about 3 days to about 5 days, or for about 3 days to about 6 days, or for about 3 days to about 1 week, or for about 3 days to about 2 weeks, or for about 3 days to about 3 weeks, or for about 3 days to about 4 weeks, or for about 4 days to about 5 days, or for about 4 days to about 6 days, or for about 4 days to about 1 week, or for about 4 days to about 2 weeks, or for about 4 days to about 3 weeks, or for about 4 days to about 4 weeks, or for about 5 days to about 6 days, or for about 5 days to about 1 week, or for about 5 days to about 2 weeks, or for about 5 days to about 3 weeks, or for about 5 days to about 4 weeks, or for about 6 days to about 1 week, or for about 6 days to about 2 weeks, or for about 6 days to about 3 weeks, or for about 6 days to about 4 weeks, or for about 1 week to about 2 weeks, or for about 1 week to about 3 weeks, or for about 1 week to about 4 weeks, or for about 2 weeks to about 3 weeks, or for about 2 weeks to about 4 weeks, or for about 3 weeks to about 4 weeks prior to transplantation of the graft.

The immune suppressant drug therapy may be discontinued after a time sufficient for implantation of the graft in the subject in need thereof. The period after which the immune suppressant drug therapy is discontinued, or in other words the time sufficient for implantation may be from at least about 1 hour, 2 hours, 6 hours, 12 hours, 1 day, 2 days, 3 days, 4 days, 5 days, 6 days, 1 week, 2 weeks, 3 weeks or 4 weeks, or for about 1 hour to about 2 hours, or for about 1 hour to about 6 hours, or for about 1 hour to about 12 hours, or for about 1 hour to about 1 day, or for about 1 hour to about 2 days, or for about 1 hour to about 3 days, or for about 1 hour to about 4 days, or for about 1 hour to about 5 days, or for about 1 hour to about 6 days, or for about 1 hour to about 1 week, or for about 1 hour to about 2 weeks, or for about 1 hour to about 3 weeks, or for about 1 hour to about 4 weeks, or for about 2 hours to about 6 hours, or for about 2 hours to about 12 hours, or for about 2 hours to about 1 day, or for about 2 hours to about 2 days, or for about 2 hours to about 3 days, or for about 2 hours to about 4 days, or for about 2 hours to about 5 days, or for about 2 hours to about 6 days, or for about 2 hours to about 1 week, or for about 2 hours to about 2 weeks, or for about 2 hours to about 3 weeks, or for about 2 hours to about 4 weeks, or for about 3 hours to about 12 hours, or for about 3 hours to about 1 day, or for about 3 hours to about 2 days, or for about 3 hours to about 3 days, or for about 3 hours to about 4 days, or for about 3 hours to about 5 days, or for about 3 hours to about 6 days, or for about 3 hours to about 1 week, or for about 3 hours to about 2 weeks, or for about 3 hours to about 3 weeks, or for about 3 hours to about 4 weeks, or for about 6 hours to about 12 hours, or for about 6 hours to about 1 day, or for about 6 hours to about 2 days, or for about 6 hours to about 3 days, or for about 6 hours to about 4 days, or for about 6 hours to about 5 days, or for about 6 hours to about 6 days, or for about 6 hours to about 1 week, or for about 6 hours to about 2 weeks, or for about 6 hours to about 3 weeks, or for about 6 hours to about 4 weeks, or for about 12 hours to about 1 day, or for about 12 hours to about 2 days, or for about 12 hours to about 3 days, or for about 12 hours to about 4 days, or for about 12 hours to about 5 days, or for about 12 hours to about 6 days, or for about 12 hours to about 1 week, or for about 12 hours to about 2 weeks, or for about 12 hours to about 3 weeks, or for about 12 hours to about 4 weeks, or for about 1 day to about 2 days, or for about 1 day to about 3 days, or for about 1 day to about 4 days, or for about 1 day to about 5 days, or for about 1 day to about 6 days, or for about 1 day to about 1 week, or for about 1 day to about 2 weeks, or for about 1 day to about 3 weeks, or for about 1 day to about 4 weeks, or for about 2 days to about 3 days, or for about 2 days to about 4 days, or for about 2 days to about 5 days, or for about 2 days to about 6 days, or for about 2 days to about 1 week, or for about 2 days to about 2 weeks, or for about 2 days to about 3 weeks, or for about 2 days to about 4 weeks, or for about 3 days to about 4 days, or for about 3 days to about 5 days, or for about 3 days to about 6 days, or for about 3 days to about 1 week, or for about 3 days to about 2 weeks, or for about 3 days to about 3 weeks, or for about 3 days to about 4 weeks, or for about 4 days to about 5 days, or for about 4 days to about 6 days, or for about 4 days to about 1 week, or for about 4 days to about 2 weeks, or for about 4 days to about 3 weeks, or for about 4 days to about 4 weeks, or for about 5 days to about 6 days, or for about 5 days to about 1 week, or for about 5 days to about 2 weeks, or for about 5 days to about 3 weeks, or for about 5 days to about 4 weeks, or for about 6 days to about 1 week, or for about 6 days to about 2 weeks, or for about 6 days to about 3 weeks, or for about 6 days to about 4 weeks, or for about 1 week to about 2 weeks, or for about 1 week to about 3 weeks, or for about 1 week to about 4 weeks, or for about 2 weeks to about 3 weeks, or for about 2 weeks to about 4 weeks, or for about 3 weeks to about 4 weeks after transplantation of the graft.

According to another embodiment, the subject in need thereof may be a subject receiving no immune suppressant drug therapy to inhibit or prevent immune rejection against the graft.

For example, the subject in need thereof receiving no immune suppressant drug therapy may be an immunocompromised subject, or a subject having a weak immune system. Examples of persons with weakened immune systems according to this embodiment include those with HIV/AIDS; cancer; and those with inherited diseases that affect the immune system (e.g., congenital agammaglobulinemia, congenital IgA deficiency). The risk of developing severe disease may differ depending on each person's degree of immune suppression.

According to another embodiment, there is provided a gem-difluorinated C-glycopeptide compound of general formula I, or a pharmaceutically acceptable base, addition salt with an acid, hydrate or solvate of the compound of general formula I for use in inhibition or prevention of immune rejection against an isolated graft contacted with the compound, prior to transplantation in a subject in need thereof:

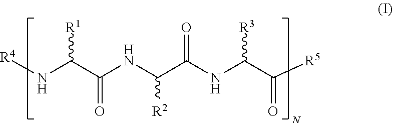

in which:
N is an integer between 1 and 5,
   R⁴═H, AA₁, or AA₁-AA₂,
   R⁵═OH, AA₁, or AA₁-AA₂,
   AA₁ and AA₂ independently represent amino acids with a non-polar side chain and
   R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

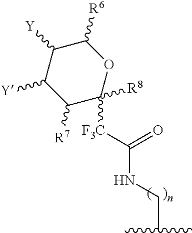

in which:
n is an integer between 3 and 4,
   Y, Y′ are independent groups
      in which Y, Y′ ═H, OR, N₃, NR′R″, or SR′″,
      where R═H, benzyl, trimethylsilyl, tert-butyldimethylsilyl, tert-butyldiphenylsilyl or acetate group,
      R′, R″ independently=H, alkyl, allyl, benzyl, tosylate group, C(═O)-alkyl or C(═O)—Bn,
      R′″═H, alkyl, or acetate group,
      R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
      R⁷═OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
      R⁸ is a hydrogen atom H or a free or protected alcohol function, and
      if R¹═R²═H, CH₃, CH₂Ph, CH(CH₃)₂, CH₂CH(CH₃)₂, or CH(CH₃)CH₂CH₃
      then R³=

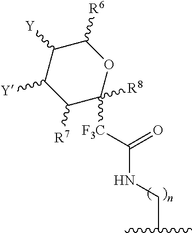

in which: n is an integer between 3 and 4,
Y, Y′ are independent groups
in which Y, Y′ ═H, OR, N₃, NR′R″, or SR′,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tertbutyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
if R¹═R³═H, CH₃, CH₂Ph, CH(CH₃)₂, CH₂CH(CH₃)₂, or CH(CH₃)CH₂CH₃
then R²=

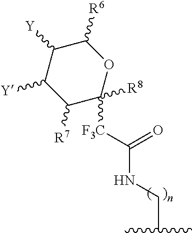

in which: n is an integer between 3 and 4,
Y, Y′ are independent groups
in which Y, Y′ ═H, OR, N₃, NR′R″, or SR′,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
if R²═R³═H, CH₃, CH(CH₃)₂, CH₂CH(CH₃)₂, or CH(CH₃)CH₂CH₃

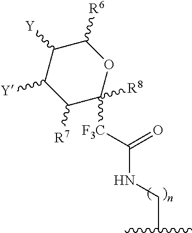

in which: n is an integer between 3 and 4,
Y, Y′ are independent groups
in which Y, Y′ H, OR, N₃, NR′R″, or SR′″,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function.

In use, the compound may be used for at least about 1 minute, or about 15 minutes, or about 30 minutes, or about 1 hour, or 6 hours, or about 12 hours, or about 24 hours, or about 48 hours, or about 72 hours, or about 1 minute to about 15 minutes, or for about 1 minute to about 30 minutes, or for about 1 minute to about 1 hour, or for about 1 minute to about 6 hours, or for about 1 minute to about 12 hours, or for about 1 minute to about 24 hours, or for about 1 minute to about 48 hours, or for about 1 minute to about 72 hours, or for about 15 minutes to about 30 minutes, or for about 15 minutes to about 1 hour, or for about 15 minutes to about 6 hours, or for about 15 minutes to about 12 hours, or for about 15 minutes to about 24 hours, or for about 15 minutes to about 48 hours, or for about 15 minutes to about 72 hours, or for about 30 minutes to about 1 hour, or for about 30 minutes to about 6 hours, or for about 30 minutes to about 12 hours, or for about 30 minutes to about 24 hours, or for about 30 minutes to about 48 hours, or for about 30 minutes to about 72 hours, or for about 1 hour to about 6 hours, or for about 1 hour to about 12 hours, or for about 1 hour to about 24 hours, or for about 1 hour to about 48 hours, or for about 1 hour to about 72 hours, or for about 2 hours to about 12 hours, or for about 2 hours to about 24 hours, or for about 2 hours to about 48 hours, or for about 2 hours to about 72 hours, or for about 6 hours to about 12 hours, or for about 6 hours to about 24 hours, or for about 6 hours to about 48 hours, or for about 6 hours to about 72 hours, or for about 12 hours to about 24 hours, or for about 12 hours to about 48 hours, or for about 12 hours to about 72 hours, or for about 24 hours to about 48 hours, or for about 24 hours to about 72 hours, or for about 24 hours to about 72 hours, or for 1 min, 15 mins, 30 mins, 1 hour, 2 hours, 6 hours, 12 hours, 24 hours, 48 hours, or 72 hours prior to transplantation.

The subject in need thereof may be under an immune suppressant drug therapy to inhibit or prevent immune rejection against the graft, for at least about 1 hour, 2 hours, 6 hours, 12 hours, 1 day, 2 days, 3 days, 4 days, 5 days, 6 days, 1 week, 2 weeks, 3 weeks or 4 weeks, or for about 1 hour to about 2 hours, or for about 1 hour to about 6 hours, or for about 1 hour to about 12 hours, or for about 1 hour to about 1 day, or for about 1 hour to about 2 days, or for about 1 hour to about 3 days, or for about 1 hour to about 4 days, or for about 1 hour to about 5 days, or for about 1 hour to about 6 days, or for about 1 hour to about 1 week, or for about 1 hour to about 2 weeks, or for about 1 hour to about 3 weeks, or for about 1 hour to about 4 weeks, or for about 2 hours to about 6 hours, or for about 2 hours to about 12 hours, or for about 2 hours to about 1 day, or for about 2 hours to about 2 days, or for about 2 hours to about 3 days, or for about 2 hours to about 4 days, or for about 2 hours to about 5 days, or for about 2 hours to about 6 days, or for about 2 hours to about 1 week, or for about 2 hours to about 2 weeks, or for about 2 hours to about 3 weeks, or for about 2 hours to about 4 weeks, or for about 3 hours to about 12 hours, or for about 3 hours to about 1 day, or for about 3 hours to about 2 days, or for about 3 hours to about 3 days, or for about 3 hours to about 4 days, or for about 3 hours to about 5 days, or for about 3 hours to about 6 days, or for about 3 hours to about 1 week, or for about 3 hours to about 2 weeks, or for about 3 hours to about 3 weeks, or for about 3 hours to about 4 weeks, or for about 6 hours to about 12 hours, or for about 6 hours to about 1 day, or for about 6 hours to about 2 days, or for about 6 hours to about 3 days, or for about 6 hours to about 4 days, or for about 6 hours to about 5 days, or for about 6 hours to about 6 days, or for about 6 hours to about 1 week, or for about 6 hours to about 2 weeks, or for about 6 hours to about 3 weeks, or for about 6 hours to about 4 weeks, or for about 12 hours to about 1 day, or for about 12 hours to about 2 days, or for about 12 hours to about 3 days, or for about 12 hours to about 4 days, or for about 12 hours to about 5 days, or for about 12 hours to about 6 days, or for about 12 hours to about 1 week, or for about 12 hours to about 2 weeks, or for about 12 hours to about 3 weeks, or for about 12 hours to about 4 weeks, or for about 1 day to about 2 days, or for about 1 day to about 3 days, or for about 1 day to about 4 days, or for about 1 day to about 5 days, or for about 1 day to about 6 days, or for about 1 day to about 1 week, or for about 1 day to about 2 weeks, or for about 1 day to about 3 weeks, or for about 1 day to about 4 weeks, or for about 2 days to about 3 days, or for about 2 days to about 4 days, or for about 2 days to about 5 days, or for about 2 days to about 6 days, or for about 2 days to about 1 week, or for about 2 days to about 2 weeks, or for about 2 days to about 3 weeks, or for about 2 days to about 4 weeks, or for about 3 days to about 4 days, or for about 3 days to about 5 days, or for about 3 days to about 6 days, or for about 3 days to about 1 week, or for about 3 days to about 2 weeks, or for about 3 days to about 3 weeks, or for about 3 days to about 4 weeks, or for about 4 days to about 5 days, or for about 4 days to about 6 days, or for about 4 days to about 1 week, or for about 4 days to about 2 weeks, or for about 4 days to about 3 weeks, or for about 4 days to about 4 weeks, or for about 5 days to about 6 days, or for about 5 days to about 1 week, or for about 5 days to about 2 weeks, or for about 5 days to about 3 weeks, or for about 5 days to about 4 weeks, or for about 6 days to about 1 week, or for about 6 days to about 2 weeks, or for about 6 days to about 3 weeks, or for about 6 days to about 4 weeks, or for about 1 week to about 2 weeks, or for about 1 week to about 3 weeks, or for about 1 week to about 4 weeks, or for about 2 weeks to about 3 weeks, or for about 2 weeks to about 4 weeks, or for about 3 weeks to about 4 weeks prior to transplantation of the graft.

The immune suppressant drug therapy may be discontinued after a time sufficient for implantation of the graft in the subject in need thereof. The period after which the immune suppressant drug therapy is discontinued, or in other words the time sufficient for implantation may be from at least about 1 hour, 2 hours, 6 hours, 12 hours, 1 day, 2 days, 3 days, 4 days, 5 days, 6 days, 1 week, 2 weeks, 3 weeks or 4 weeks, or for about 1 hour to about 2 hours, or for about 1 hour to about 6 hours, or for about 1 hour to about 12 hours, or for about 1 hour to about 1 day, or for about 1 hour to about 2 days, or for about 1 hour to about 3 days, or for about 1 hour to about 4 days, or for about 1 hour to about 5 days, or for about 1 hour to about 6 days, or for about 1 hour to about 1 week, or for about 1 hour to about 2 weeks, or for about 1 hour to about 3 weeks, or for about 1 hour to about 4 weeks, or for about 2 hours to about 6 hours, or for about 2 hours to about 12 hours, or for about 2 hours to about 1 day, or for about 2 hours to about 2 days, or for about 2 hours to about 3 days, or for about 2 hours to about 4 days, or for about 2 hours to about 5 days, or for about 2 hours to about 6 days, or for about 2 hours to about 1 week, or for about 2 hours to about 2 weeks, or for about 2 hours to about 3 weeks, or for about 2 hours to about 4 weeks, or for about 3 hours to about 12 hours, or for about 3 hours to about 1 day, or for about 3 hours to about 2 days, or for about 3 hours to about 3 days, or for about 3 hours to about 4 days, or for about 3 hours to about 5 days, or for about 3 hours to about 6 days, or for about 3 hours to about 1 week, or for about 3 hours to about 2 weeks, or for about 3 hours to about 3 weeks, or for about 3 hours to about 4 weeks, or for about 6 hours to about 12 hours, or for about 6 hours to about 1 day, or for about 6 hours to about 2 days, or for about 6 hours to about 3 days, or for about 6 hours to about 4 days, or for about 6 hours to about 5 days, or for about 6 hours to about 6 days, or for about 6 hours to about 1 week, or for about 6 hours to about 2 weeks, or for about 6 hours to about 3 weeks, or for about 6 hours to about 4 weeks, or for about 12 hours to about 1 day, or for about 12 hours to about 2 days, or for about 12 hours to about 3 days, or for about 12 hours to about 4 days, or for about 12 hours to about 5 days, or for about 12 hours to about 6 days, or for about 12 hours to about 1 week, or for about 12 hours to about 2 weeks, or for about 12 hours to about 3 weeks, or for about 12 hours to about 4 weeks, or for about 1 day to about 2 days, or for about 1 day to about 3 days, or for about 1 day to about 4 days, or for about 1 day to about 5 days, or for about 1 day to about 6 days, or for about 1 day to about 1 week, or for about 1 day to about 2 weeks, or for about 1 day to about 3 weeks, or for about 1 day to about 4 weeks, or for about 2 days to about 3 days, or for about 2 days to about 4 days, or for about 2 days to about 5 days, or for about 2 days to about 6 days, or for about 2 days to about 1 week, or for about 2 days to about 2 weeks, or for about 2 days to about 3 weeks, or for about 2 days to about 4 weeks, or for about 3 days to about 4 days, or for about 3 days to about 5 days, or for about 3 days to about 6 days, or for about 3 days to about 1 week, or for about 3 days to about 2 weeks, or for about 3 days to about 3 weeks, or for about 3 days to about 4 weeks, or for about 4 days to about 5 days, or for about 4 days to about 6 days, or for about 4 days to about 1 week, or for about 4 days to about 2 weeks, or for about 4 days to about 3 weeks, or for about 4 days to about 4 weeks, or for about 5 days to about 6 days, or for about 5 days to about 1 week, or for about 5 days to about 2 weeks, or for about 5 days to about 3 weeks, or for about 5 days to about 4 weeks, or for about 6 days to about 1 week, or for about 6 days to about 2 weeks, or for about 6 days to about 3 weeks, or for about 6 days to about 4 weeks, or for about 1 week to about 2 weeks, or for about 1 week to about 3 weeks, or for about 1 week to about 4 weeks, or for about 2 weeks to about 3 weeks, or for about 2 weeks to about 4 weeks, or for about 3 weeks to about 4 weeks after transplantation of the graft.

According to another embodiment, the subject in need thereof may be a subject receiving no immune suppressant drug therapy to inhibit or prevent immune rejection against the graft.

For example, the subject in need thereof receiving no immune suppressant drug therapy may be an immunocompromised subject, or a subject having a weak immune system. Examples of persons with weakened immune systems according to this embodiment include those with HIV/AIDS; cancer; and those with inherited diseases that affect the immune system (e.g., congenital agammaglobulinemia, congenital IgA deficiency). The risk of developing severe disease may differ depending on each person's degree of immune suppression.

According to another embodiment, there is disclosed an isolated graft contacted with a gem-difluorinated C-glycopeptide compound of general formula I, or a pharmaceutically acceptable base, addition salt with an acid, hydrate or solvate of the compound of general formula I prior to transplantation in a subject in need thereof, for inhibition or prevention of immune rejection against the isolated graft contacted with the compound:

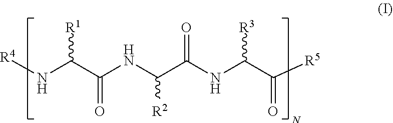

in which:
N is an integer between 1 and 5,
   R⁴═H, AA₁, or AA₁-AA₂,
   R⁵═OH, AA₁, or AA₁-AA₂,
   AA₁ and AA₂ independently represent amino acids with a non-polar side chain and
   R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

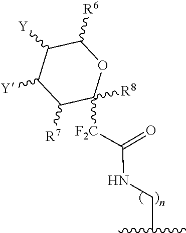

in which:
n is an integer between 3 and 4,
   Y, Y′ are independent groups
      in which Y, Y′ ═H, OR, N₃, NR′R″, or SR′″,
      where R═H, benzyl, trimethylsilyl, tert-butyldimethylsilyl, tert-butyldiphenylsilyl or acetate group,
      R′, R″ independently=H, alkyl, allyl, benzyl, tosylate group, C(═O)-alkyl or C(═O)—Bn,
      R′″═H, alkyl, or acetate group,
      R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
      R⁷═OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
      R⁸ is a hydrogen atom H or a free or protected alcohol function, and
      if R¹═R²═H, CH₃, CH₂Ph, CH(CH₃)₂, CH₂CH(CH₃)₂, or CH(CH₃)CH₂CH₃
      then R³=

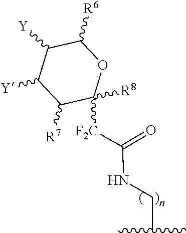

in which: n is an integer between 3 and 4,
Y, Y′ are independent groups
in which Y, Y′ ═H, OR, N₃, NR′R″, or SR′,
   where R═H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″═H, alkyl, or acetate group,
   R⁶ is H, CH₃, CH₂OH, CH₂-glycoside group, or CH₂-OGP in which GP is a protector group selected from alkyl, benzyl, trimethylsilyl, tertbutyldimethylsilyl, tert-butyldiphenylsilyl, or acetate group,
   R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
   R⁸ is a hydrogen atom H or a free or protected alcohol function,
   if R¹═R³═H, CH₃, CH₂Ph, CH(CH₃)₂, CH₂CH(CH₃)₂, or CH(CH₃)CH₂CH₃
   then R²=

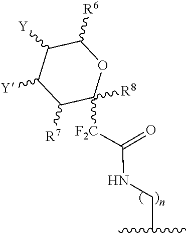

in which: n is an integer between 3 and 4,
Y, Y′ are independent groups
in which Y, Y′ ═H, OR, N₃, NR′R″, or SR′,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
if R²═R³═H, CH₃, CH(CH₃)₂, CH₂CH(CH₃)₂, or CH(CH₃)CH₂CH₃
then R¹=

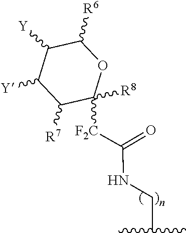

in which: n is an integer between 3 and 4,
Y, Y′ are independent groups in which Y, Y′ H, OR, N₃, NR′R″, or SR′″,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″═H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function.

In use, the compound may be used for at least about 1 minute, or about 15 minutes, or about 30 minutes, or about 1 hour, or 6 hours, or about 12 hours, or about 24 hours, or about 48 hours, or about 72 hours, or about 1 minute to about 15 minutes, or for about 1 minute to about 30 minutes, or for about 1 minute to about 1 hour, or for about 1 minute to about 6 hours, or for about 1 minute to about 12 hours, or for about 1 minute to about 24 hours, or for about 1 minute to about 48 hours, or for about 1 minute to about 72 hours, or for about 15 minutes to about 30 minutes, or for about 15 minutes to about 1 hour, or for about 15 minutes to about 6 hours, or for about 15 minutes to about 12 hours, or for about 15 minutes to about 24 hours, or for about 15 minutes to about 48 hours, or for about 15 minutes to about 72 hours, or for about 30 minutes to about 1 hour, or for about 30 minutes to about 6 hours, or for about 30 minutes to about 12 hours, or for about 30 minutes to about 24 hours, or for about 30 minutes to about 48 hours, or for about 30 minutes to about 72 hours, or for about 1 hour to about 6 hours, or for about 1 hour to about 12 hours, or for about 1 hour to about 24 hours, or for about 1 hour to about 48 hours, or for about 1 hour to about 72 hours, or for about 2 hours to about 12 hours, or for about 2 hours to about 24 hours, or for about 2 hours to about 48 hours, or for about 2 hours to about 72 hours, or for about 6 hours to about 12 hours, or for about 6 hours to about 24 hours, or for about 6 hours to about 48 hours, or for about 6 hours to about 72 hours, or for about 12 hours to about 24 hours, or for about 12 hours to about 48 hours, or for about 12 hours to about 72 hours, or for about 24 hours to about 48 hours, or for about 24 hours to about 72 hours, or for about 24 hours to about 72 hours, or for 1 min, 15 mins, 30 mins, 1 hour, 2 hours, 6 hours, 12 hours, 24 hours, 48 hours, or 72 hours prior to transplantation.

The subject in need thereof may be under an immune suppressant drug therapy to inhibit or prevent immune rejection against the graft, for at least about 1 hour, 2 hours, 6 hours, 12 hours, 1 day, 2 days, 3 days, 4 days, 5 days, 6 days, 1 week, 2 weeks, 3 weeks or 4 weeks, or for about 1 hour to about 2 hours, or for about 1 hour to about 6 hours, or for about 1 hour to about 12 hours, or for about 1 hour to about 1 day, or for about 1 hour to about 2 days, or for about 1 hour to about 3 days, or for about 1 hour to about 4 days, or for about 1 hour to about 5 days, or for about 1 hour to about 6 days, or for about 1 hour to about 1 week, or for about 1 hour to about 2 weeks, or for about 1 hour to about 3 weeks, or for about 1 hour to about 4 weeks, or for about 2 hours to about 6 hours, or for about 2 hours to about 12 hours, or for about 2 hours to about 1 day, or for about 2 hours to about 2 days, or for about 2 hours to about 3 days, or for about 2 hours to about 4 days, or for about 2 hours to about 5 days, or for about 2 hours to about 6 days, or for about 2 hours to about 1 week, or for about 2 hours to about 2 weeks, or for about 2 hours to about 3 weeks, or for about 2 hours to about 4 weeks, or for about 3 hours to about 12 hours, or for about 3 hours to about 1 day, or for about 3 hours to about 2 days, or for about 3 hours to about 3 days, or for about 3 hours to about 4 days, or for about 3 hours to about 5 days, or for about 3 hours to about 6 days, or for about 3 hours to about 1 week, or for about 3 hours to about 2 weeks, or for about 3 hours to about 3 weeks, or for about 3 hours to about 4 weeks, or for about 6 hours to about 12 hours, or for about 6 hours to about 1 day, or for about 6 hours to about 2 days, or for about 6 hours to about 3 days, or for about 6 hours to about 4 days, or for about 6 hours to about 5 days, or for about 6 hours to about 6 days, or for about 6 hours to about 1 week, or for about 6 hours to about 2 weeks, or for about 6 hours to about 3 weeks, or for about 6 hours to about 4 weeks, or for about 12 hours to about 1 day, or for about 12 hours to about 2 days, or for about 12 hours to about 3 days, or for about 12 hours to about 4 days, or for about 12 hours to about 5 days, or for about 12 hours to about 6 days, or for about 12 hours to about 1 week, or for about 12 hours to about 2 weeks, or for about 12 hours to about 3 weeks, or for about 12 hours to about 4 weeks, or for about 1 day to about 2 days, or for about 1 day to about 3 days, or for about 1 day to about 4 days, or for about 1 day to about 5 days, or for about 1 day to about 6 days, or for about 1 day to about 1 week, or for about 1 day to about 2 weeks, or for about 1 day to about 3 weeks, or for about 1 day to about 4 weeks, or for about 2 days to about 3 days, or for about 2 days to about 4 days, or for about 2 days to about 5 days, or for about 2 days to about 6 days, or for about 2 days to about 1 week, or for about 2 days to about 2 weeks, or for about 2 days to about 3 weeks, or for about 2 days to about 4 weeks, or for about 3 days to about 4 days, or for about 3 days to about 5 days, or for about 3 days to about 6 days, or for about 3 days to about 1 week, or for about 3 days to about 2 weeks, or for about 3 days to about 3 weeks, or for about 3 days to about 4 weeks, or for about 4 days to about 5 days, or for about 4 days to about 6 days, or for about 4 days to about 1 week, or for about 4 days to about 2 weeks, or for about 4 days to about 3 weeks, or for about 4 days to about 4 weeks, or for about 5 days to about 6 days, or for about 5 days to about 1 week, or for about 5 days to about 2 weeks, or for about 5 days to about 3 weeks, or for about 5 days to about 4 weeks, or for about 6 days to about 1 week, or for about 6 days to about 2 weeks, or for about 6 days to about 3 weeks, or for about 6 days to about 4 weeks, or for about 1 week to about 2 weeks, or for about 1 week to about 3 weeks, or for about 1 week to about 4 weeks, or for about 2 weeks to about 3 weeks, or for about 2 weeks to about 4 weeks, or for about 3 weeks to about 4 weeks prior to transplantation of the graft.

The immune suppressant drug therapy may be discontinued after a time sufficient for implantation of the graft in the subject in need thereof. The period after which the immune suppressant drug therapy is discontinued, or in other words the time sufficient for implantation may be from at least about 1 hour, 2 hours, 6 hours, 12 hours, 1 day, 2 days, 3 days, 4 days, 5 days, 6 days, 1 week, 2 weeks, 3 weeks or 4 weeks, or for about 1 hour to about 2 hours, or for about 1 hour to about 6 hours, or for about 1 hour to about 12 hours, or for about 1 hour to about 1 day, or for about 1 hour to about 2 days, or for about 1 hour to about 3 days, or for about 1 hour to about 4 days, or for about 1 hour to about 5 days, or for about 1 hour to about 6 days, or for about 1 hour to about 1 week, or for about 1 hour to about 2 weeks, or for about 1 hour to about 3 weeks, or for about 1 hour to about 4 weeks, or for about 2 hours to about 6 hours, or for about 2 hours to about 12 hours, or for about 2 hours to about 1 day, or for about 2 hours to about 2 days, or for about 2 hours to about 3 days, or for about 2 hours to about 4 days, or for about 2 hours to about 5 days, or for about 2 hours to about 6 days, or for about 2 hours to about 1 week, or for about 2 hours to about 2 weeks, or for about 2 hours to about 3 weeks, or for about 2 hours to about 4 weeks, or for about 3 hours to about 12 hours, or for about 3 hours to about 1 day, or for about 3 hours to about 2 days, or for about 3 hours to about 3 days, or for about 3 hours to about 4 days, or for about 3 hours to about 5 days, or for about 3 hours to about 6 days, or for about 3 hours to about 1 week, or for about 3 hours to about 2 weeks, or for about 3 hours to about 3 weeks, or for about 3 hours to about 4 weeks, or for about 6 hours to about 12 hours, or for about 6 hours to about 1 day, or for about 6 hours to about 2 days, or for about 6 hours to about 3 days, or for about 6 hours to about 4 days, or for about 6 hours to about 5 days, or for about 6 hours to about 6 days, or for about 6 hours to about 1 week, or for about 6 hours to about 2 weeks, or for about 6 hours to about 3 weeks, or for about 6 hours to about 4 weeks, or for about 12 hours to about 1 day, or for about 12 hours to about 2 days, or for about 12 hours to about 3 days, or for about 12 hours to about 4 days, or for about 12 hours to about 5 days, or for about 12 hours to about 6 days, or for about 12 hours to about 1 week, or for about 12 hours to about 2 weeks, or for about 12 hours to about 3 weeks, or for about 12 hours to about 4 weeks, or for about 1 day to about 2 days, or for about 1 day to about 3 days, or for about 1 day to about 4 days, or for about 1 day to about 5 days, or for about 1 day to about 6 days, or for about 1 day to about 1 week, or for about 1 day to about 2 weeks, or for about 1 day to about 3 weeks, or for about 1 day to about 4 weeks, or for about 2 days to about 3 days, or for about 2 days to about 4 days, or for about 2 days to about 5 days, or for about 2 days to about 6 days, or for about 2 days to about 1 week, or for about 2 days to about 2 weeks, or for about 2 days to about 3 weeks, or for about 2 days to about 4 weeks, or for about 3 days to about 4 days, or for about 3 days to about 5 days, or for about 3 days to about 6 days, or for about 3 days to about 1 week, or for about 3 days to about 2 weeks, or for about 3 days to about 3 weeks, or for about 3 days to about 4 weeks, or for about 4 days to about 5 days, or for about 4 days to about 6 days, or for about 4 days to about 1 week, or for about 4 days to about 2 weeks, or for about 4 days to about 3 weeks, or for about 4 days to about 4 weeks, or for about 5 days to about 6 days, or for about 5 days to about 1 week, or for about 5 days to about 2 weeks, or for about 5 days to about 3 weeks, or for about 5 days to about 4 weeks, or for about 6 days to about 1 week, or for about 6 days to about 2 weeks, or for about 6 days to about 3 weeks, or for about 6 days to about 4 weeks, or for about 1 week to about 2 weeks, or for about 1 week to about 3 weeks, or for about 1 week to about 4 weeks, or for about 2 weeks to about 3 weeks, or for about 2 weeks to about 4 weeks, or for about 3 weeks to about 4 weeks after transplantation of the graft.

According to another embodiment, the subject in need thereof may be a subject receiving no immune suppressant drug therapy to inhibit or prevent immune rejection against the graft.

For example, the subject in need thereof receiving no immune suppressant drug therapy may be an immunocompromised subject, or a subject having a weak immune system. Examples of persons with weakened immune systems according to this embodiment include those with HIV/AIDS; cancer; and those with inherited diseases that affect the immune system (e.g., congenital agammaglobulinemia, congenital IgA deficiency). The risk of developing severe disease may differ depending on each person's degree of immune suppression.

The invention includes the compounds as shown, and also includes (where possible) individual diastereomers, enantiomers, and epimers of the compounds, and mixtures of diastereomers and/or enantiomers thereof including racemic mixtures. Although the specific stereochemistries disclosed herein are preferred, other stereoisomers, including diastereomers, enantiomers, epimers, and mixtures of these may also be useful. Inactive or less active diastereoisomers and enantiomers are useful for scientific studies relating to the targets and/or the mechanism of activation.

The compounds disclosed herein may be used in pharmaceutical compositions comprising (a) the compound(s) or pharmaceutically acceptable salts thereof, and (b) a pharmaceutically acceptable carrier. The compounds may be used in pharmaceutical compositions that include one or more other active pharmaceutical ingredients. The compounds may also be used in pharmaceutical compositions in which the compound of Formula I, II or Ill, or a pharmaceutically acceptable salt thereof is the only active ingredient.

Compounds of structural Formula I, structural Formula II and/or structural Formula III may contain one or more asymmetric centers and can thus occur as racemates and racemic mixtures, single enantiomers, diastereomeric mixtures and individual diastereomers. The present invention is meant to comprehend all such isomeric forms of the compounds of structural Formula I, structural Formula II and/or structural Formula III.

Compounds of structural Formula I, structural Formula II and/or structural Formula III may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general structural Formula I, structural Formula II and/or structural Formula III may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and unless specified otherwise, are meant to include both E and Z geometric isomers.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

In the compounds of generic Formula I, Formula II and/or Formula III, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I, Formula II and/or Formula III. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I, Formula II and/or Formula III can be prepared without undue experimentation by conventional techniques well known to those skilled in the art.

### Salts and Formulations

It will be understood that, as used herein, references to the compounds of structural Formula I, Formula II and/or Formula III are meant to also include the pharmaceutically acceptable salts, and also salts that are not pharmaceutically acceptable when they are used as precursors to the free compounds or their pharmaceutically acceptable salts or in other synthetic manipulations. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, edetate, edisylate, estolate, esylate, fumarate, gluceptate, gluconate, glutamate, hexylresorcinate, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as acetyl, pivaloyl, benzoyl, and aminoacyl, can be employed. Included are those esters and acyl groups known in the art for modifying the solubility or hydrolysis characteristics for use as sustained-release or prodrug formulations.

Solvates, in particular hydrates, of the compounds of structural Formula I, Formula II and/or Formula III are included in the present invention as well.

According to an embodiment, the compounds of structural Formula I, Formula II and/or Formula III may be included in various formulations for use as medicaments.

Aqueous suspensions contain the active material in admixture with excipients suitable for the manufacture of aqueous suspensions. Such excipients are suspending agents, for example sodium carboxymethyl-cellulose, methylcellulose, hydroxypropylmethy-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the the partial esters with ethylene oxide, for example polyoxyethylene sorbitan monooleate. The emulsions may also contain sweetening and flavouring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

According to an embodiment, the cells are isolated using methods known in the art for their preparation. For example, the cells may be isolated from donors using mixtures of enzymes such as Collagenase I and Collagenase II, Thermolysin, non-clostridial neutral protease, or other enzymes being used for such purpose. The isolated cells may then be cultured under normal tissue culture conditions in standard tissue culture flasks.

According to an embodiment, the neurosensory precursor cells may be treated with a gem-difluorinated C-glycopeptide compound of general formula I—preferably, the compound of Formula II, and most preferably the compound of formula III in concentrations varying from about 0.01 mg/ml to about 5 mg/ml; or from about 0.1 mg/ml to about 5 mg/ml; or from about 0.5 mg/ml to about 5 mg/ml; or from about 1 mg/ml to about 5 mg/ml; or from about 3 mg/ml to about 5 mg/ml; or from about 0.01 mg/ml to about 3 mg/ml, or from about 0.1 mg/ml to about 3 mg/ml, or from about 0.5 mg/ml to about 3 mg/ml, or from about 1 mg/ml to about 3 mg/ml, or from about 0.01 mg/ml to about 1 mg/ml; or from about 0.1 mg/ml to about 1 mg/ml; or from about 0.5 mg/ml to about 1 mg/ml; or from about 0.01 mg/ml to about 0.5 mg/ml; or from about 0.1 mg/ml to about 0.5 mg/ml; or from about 0.01 mg/ml to about 0.1 mg/ml; or about 3 mg/ml. According to embodiments, the amounts above are considered to be therapeutically effective amounts for the purpose of the present inventions.

According to another embodiment, the cells are contacted with the gem-difluorinated C-glycopeptide compound for a time sufficient to effect improvements on cell viability and survival rate. According to embodiments, the time sufficient may be from about 12 hours to 120 hours, or from about 12 hours to about 96 hours, or from about 12 hours to about 72 hours, or from about 12 hours to about 48 hours, or from about 12 hours to about 24 hours, or about 120 hours, or about 96 hours, or about 72 hours, or about 48 hours, or about 24 hours, or about 12 hours, or about 10 hours, or about 8 hours, or about 6 hours, or about 4 hours, or about 2 hours, or about 1 hour. In embodiments, the wherein the isolated neurosensory precursor cell is contacted with the compound for 1 hour, 55 mins, 50 mins, 45 mins, 40 mins, 35 mins, 30 mins, 25 mins, 20 mins, 15 mins, 10 mins, 5 mins, 4 mins, 3 mins, 2 mins, 1 mins, 45 secs, or 30 secs, or at least 1 hour, or at least 55 mins, or at least 50 mins, or at least 45 mins, or at least 40 mins, or at least 35 mins, or at least 30 mins, or at least 25 mins, or at least 20 mins, or at least 15 mins, or at least 10 mins, or at least 5 mins, or at least 4 mins, or at least 3 mins, or at least 2 mins, or at least 1 mins, or at least 45 secs, or at least 30 secs.

In another embodiment there is disclosed a cell preparation prepared according to the method of the present invention, in a pharmaceutically acceptable carrier. According to an embodiment, the cell preparation may be used for the preparation of a medicament for a cell transplantation. According to another embodiment, the cell preparation may be used for a cell transplantation.

In another embodiment, there is disclosed a method of transplantation comprising transplanting a cell preparation of the present invention to a subject in need thereof. The subject may be a mammal, and preferably a human.

The present invention will be more readily understood by referring to the following examples which are given to illustrate the invention rather than to limit its scope.

### Example 1

The objective of this study was to determine the effect of 24-hour pretreatment with anti-aging glycoprotein (AAGP™) at 4 mg/mL on the long-term (6 months) survival and functional activity of photoreceptor precursor cells (PPCs) following their subretinal transplantation into the eye of rabbits with retinal degeneration.

| | |
|---|---|
| AAGP ™ | Anti-aging glycoprotein |
| DMEM/F12 | Dulbecco's modified Eagle's medium/Nutrient Mixture F12 |
| EB | Embryoid bodies |
| ERG | Electroretinogram |
| GCL | Ganglion cell layer |
| hESC | Human embryonic stem cells |
| ID | Identification |
| IGF-1 | Insulin-like growth factor 1 |
| INL | Inner nuclear layer |
| ONL | Outer nuclear layer |
| PBS | Phosphate buffered saline |
| PPCs | Photoreceptor precursor cells |
| RD | Retinal degeneration |

In vivo system
Species: Rabbit (Oryctolagus cuniculus)
Strain: White albino New Zealand, Crl: KBL (NZW) BR
Source: Charles River Canada Inc
   324 St-Regis Nord, C.P. 400
   Saint Constant, Quebec, Canada
Sex: Female
Age: 5 weeks old (upon arrival to test facility)
Weight: 1.25 to 1.50 kg (upon arrival to test facility)

9 New Zealand rabbits were obtained from Charles River at the age of 5 weeks, weighing 1.25 to 1.50 kg. Animals were maintained on a 12 h light/dark cycle and research carried out in accordance with protocols compliant to the Canadian Council on Animal Care with the approval of the Animal Care Committee at the University of British Columbia and with the Association for Research in Vison and Ophthalmology statement for the use of animals in vision research.

Species Identification and Acclimation

Animals were acclimated to laboratory conditions for at least 5 days before being placed into different study groups. Each animal was uniquely identified with identification (ID) numbers on their ears by using permanent markers.

Experimental Design

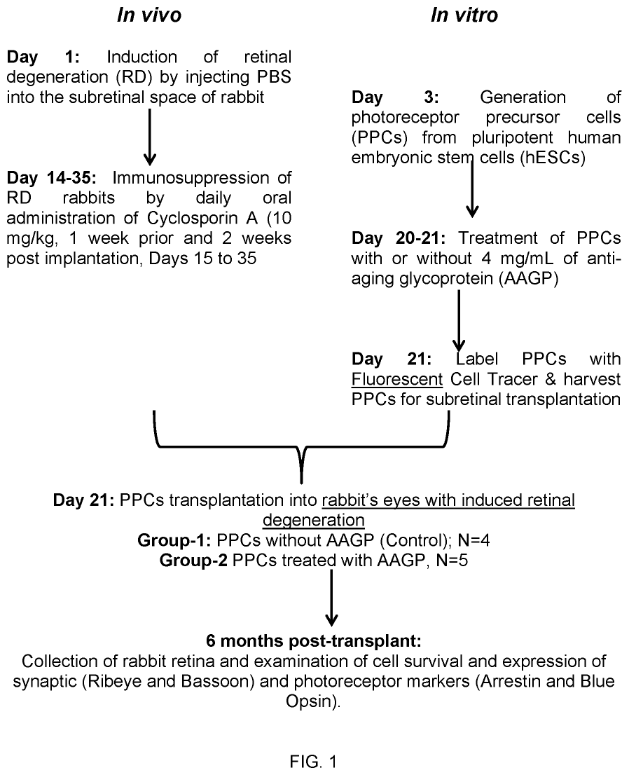

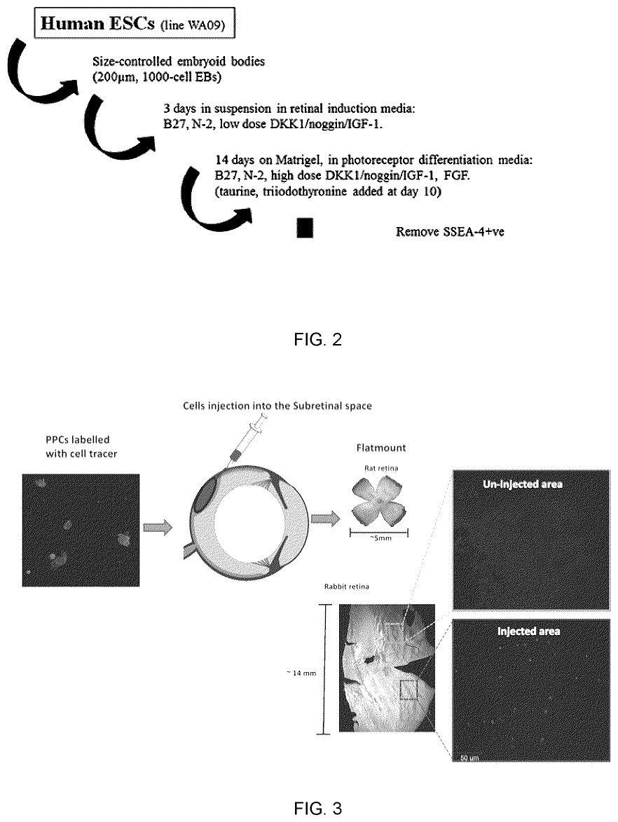

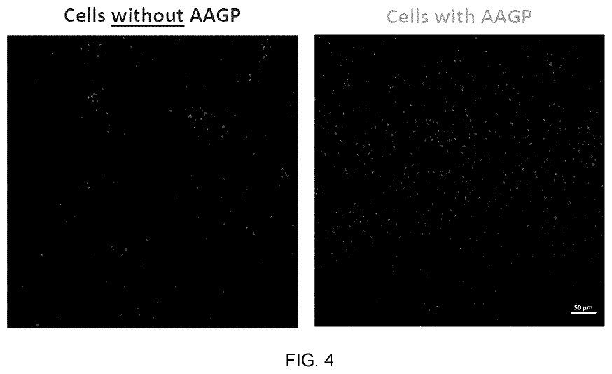

PPCs were generated in vitro from pluripotent human embryonic stem cells (hESCs) and incubated for 24 hours in media alone (control group) or media containing 4 mg/mL AAGP™ Following incubation, treated and untreated PPCs were labeled and injected into the subretinal space of the eye of immunosuppressed rabbits with induced retinal degeneration (4-5 rabbits/group). Animals were euthanized 6 month later, retinas were collected, and cell survival was determined in excised retinal flatmounts by imaging with confocal laser scanning system. The functional activity of transplanted cells was assessed by immunostaining for synaptic (Ribeye and Bassoon) and photoreceptor markers (Arrestin and Blue Opsin). The study design summary is presented in the table and flowchart presented in FIG. 1.

TABLE 1
Study design. a -Treatment duration was 24 hours (immediately prior to transplantation).

| | | AAGP | | |
|---|---|---|---|---|
| | Animal | concentration | PPC | histopathology and |
| Group | number/group | (mg/mL)^{a} | transplantation | immunostaining |
| PPCs control (no AAGP) | 4 | 0 | 200,000 cells | 6 months |
| AAGP-treated PPCs | 5 | 4 | 200,000 cells | 6 months |

Procedures

Induction of Retinal Degeneration

Retinal degeneration was induced on Day 1 of the study. Animals were put under general anesthesia by intramuscular administration of 20 mg/kg ketamine (Narketan™, 100 mg/mL; Vetoquinol N.-A. Inc. Lavaltrie, QC, Canada) and 5 mg/kg xylazine (Rompun™, 20 mg/mL; Bayer Inc. Toronto, Canada), and the pupils were dilated with 1% tropicamide (Bausch and Lomb, Rochester, N.Y., USA). Povidone-iodine (Alcon) antimicrobial solution was used to wash the eye, lids, surrounding skin, and fur of the recipient. Tear-gel (Alcon) was applied to the cornea throughout the surgery.

Phosphate buffered saline (PBS) (Life Technologies Inc, Toronto, Canada) was drawn into a 1 mL syringe connected with 30-G needle (BD, Canada). 250 μL PBS was injected slowly, subretinally, forming a uniform bleb that was clearly visible under the operating microscope. Care was taken to maintain the tip within the bleb during the injection to minimize reflux. Retinal degeneration was induced in one eye of each animal while leaving the other eye intact.

Postsurgical topical antibiotics (1% Chloramphenicol eye ointment, Netcare Healthcare UK Ltd, London) were given. Upon postsurgical follow-up, none of the eyes showed signs of extra- or intraocular infection or inflammation.

Immunosuppression

Transplant recipients underwent daily oral administration of the immunosuppressant, cyclosporine-A (Neoral™, Novartis, East Hanover, N.J., USA) at 10 mg/kg/day, for 1 week prior to transplantation and two weeks post transplantation and administration was discontinued afterward. Cyclosporine A was administered by oral gavage.

Preparation of PPCs

Photoreceptor precursor cells (PPCs) were derived from pluripotent human ES cells (hESC) line WA09 (Wi Cell) as per the protocol by Yanai et al., 2013 and Yanai et al., 2015.

1) hESCs, line WA09, were cultured in complete TeSR™2 growth medium (StemCell Technologies) in a 37° C. incubator in 5% C02 and humidity which was provided by distilled H₂O in a tray at bottom of the incubator [1, 2]. Cells were cultured in 6-well plates (Nunclon Delta Surface, Thermo Scientific) coated with growth factor-reduced Matrigel (BD biosciences).

hESCs were differentiated into PPCs through a multistep procedure as shown in the Figure below, described in detail in Yanai et al., 2013 and Yanai et al., 2015 and in sections below.

2) To generate size-controlled embryoid bodies (EBs), hESCs were washed with DMEM/F12 (Life Technologies), dissociated into single cells using 0.75 mL of Accutase™ (StemCell Technologies) per well. Cells were incubated for 6-10 min at 37° C., until most cells were dislodged, and then collected with 4 mL/well of DMEM/F12, centrifuged for 5 minutes at 300 g and the pellet resuspended in EB formation media [TeSR™2 supplemented with 10 μM Rho-Associated Coil Kinase (ROCK) inhibitor (StemCell Technologies)]. Viable cells were counted with trypan blue and 1.2×10⁶ viable hESCs were seeded into each well of the AggreWell400™ plate (StemCell Technologies). The plate was centrifuged for 3 min at 100 g and placed into a 37° C. incubator for 24 hours.

3) Following incubation, EBs were harvested by gentle pipetting, passed through a 37 μm cell strainer (StemCell Technologies) to discard unincorporated single cells and distributed into ultra-low attachment 24-well dishes (Corning). For the next three days EBs were maintained in EB resuspension medium, also called retinal induction medium [(DMEM/F12, 10% knockout serum replacement, 2% custom B-27 and 1% N-2 supplements (Life Technologies), 1 ng/mL recombinant human DKK-1, 1 ng/mL mouse noggin and 5 ng/mL recombinant human insulin-like growth factor (IGF-1) (R&D Systems)].

4) On the fourth day, EBs were collected into a 50 ml tube, centrifuged for 5 minutes at 100 g, re-suspended in PPC differentiation medium [DMEM/F12, 2% custom B-27 and 1% N-2 supplements (Life Technologies), 10 ng/mL mouse noggin, 10 ng/mL recombinant human Dkk-1, 10 ng/mL recombinant human IGF-1 and 5 ng/mL recombinant human basic FGF (Life Technologies)] and plated on Matrigel coated 6-well dishes (same dishes as above). Culture medium was replaced every other day. On incubation days 10-17 (including), PPC differentiation medium was supplemented with taurine (Sigma) and triiodothyronine (T3, Sigma) at 20 mM and 40 ng/mL, respectively, and feeding continued at the same schedule.

Treatment of PPCs with AAGP™

24 hours prior to cell transplantation PPCs were treated with 4 mg/mL of AAGP™ prepared on the day of treatment by dissolving the required amount of AAGP™ powder in basic cell culture medium (DMEM/F12, 2% B-27 and 1% N-2 supplements, 1% Sodium Pyruvate and 1% Non-essential amino acids). All components were purchased from Life Technologies. The pH was adjusted by adding 1N NaOH. Whatman pH indicator paper (ranges pH 4.5 to 10) was used to check the medium pH. Following pH adjustment, AAGP™ containing medium was filter sterilized (0.22 μm Millipore syringe filter).

PPC culture medium was removed and replaced with culture medium containing 4 mg/mL AAGP™. The control group of cells received the same basic culture medium without AAGP™. Cell culture plates were incubated at 37° C. with 5% C02 and humidity which was provided by distilled H₂O in a tray at bottom of the incubator.

Cell Labelling and Transplantation

Following 24-hour incubation with or without AAGP™, PPCs were labeled with Cell Trace Far Red DDAO-SE according to manufacturer specifications (Life Technologies). After labeling, cells were dissociated using TrypLE™ Express Enzyme (Life Technologies). Basic cell culture medium was aspirated and saved. 1 mL of TrypLE™ was added to each well and cells were incubated for 3 min after which the collected culture media was added back to the wells to neutralize trypsin. Cells were collected, centrifuged for 5 min at 1500 rpm, supernatants were discarded, and cells were re-suspended in sterile Dulbecco PBS (Life Technologies).

Cell viability was determined by Trypan blue. Labeled cells were adjusted to a concentration of 10⁶ viable cells/mL and kept on ice prior to transplantation.

200,000 cells were aseptically injected into the sub-retinal space where retinal degeneration was previously induced. Animal handling and injection procedures were the same as described in section “Induction of retinal degeneration” above.

Tissue processing, Immunostaining and Confocal Imajin

Animals were euthanized 6 months after cell transplantation. Following euthanasia, recipient eyes were marked at the inferior midline and nasal equator with Blue dye for orientation then enucleated and fixed in 4% paraformaldehyde for 24 h at 4° C. This was followed by cryopreservation treatments consisting of three 5-min washes in PBS and incubation with 30% sucrose (in PBS) for 24 h at 4° C. Neurosensory retina flatmounts were obtained by blunt dissection of retina from these eyecups, flattening the tissue onto Superfrost™ Plus microscope slides (Fisher), and mounting under a coverslip with DAPI Fluoromount-G™ (Southern Biotech). PPCs were visualized by fluorescent microscopy and their numbers were assessed qualitatively by visual examination.

Following flatmount preparation, immunostaining for synaptic markers (Ribeye and Bassoon) and photoreceptor markers (Arrestin and Blue Opsin) was performed as per standard protocol. Tissues were re-fixed with 4% paraformaldehyde for 5 min at room temperature and cryopreserved with 30% sucrose (in PBS) for overnight at 4° C., and then embedded in Polyfreeze™ medium (Polysciences). Immunolabelling was carried out using ˜10 μm thick frozen retinal sections placed on Superfrost™ Plus microscope slides. Sections were air dried for 1 hour at room temperature and blocked with blocking buffer (2% normal goat serum, 0.1% Triton X-100 in PBS) for 1 hour at room temperature. After this, sections were incubated overnight at 4° C. with the appropriate primary antibody diluted in blocking buffer.

After extensive washes in 0.1% Tween 20 in PBS (3 times for 10 minutes each) sections were incubated for 1 hour with the appropriate fluorescent secondary antibody diluted in PBS containing 0.1% Tween 20. Following 3 washes in 0.1% Tween 20 in PBS (as described above), nuclei were counter stained with DAPI Fluoromount-G. Confocal images were acquired using a Zeiss LSM 510 META confocal laser scanning system.

The following staining and imaging conditions were used for each marker:

Arrestin Staining

Primary antibody: Anti-Arrestin (Cat #MAB5580; MILLIPORE; Dilution 1:500, overnight incubation at 4° C.).

Secondary antibody: Goat anti-Mouse IgG (H+L) Cross-Adsorbed Secondary Antibody, Alexa Fluor 488 (Cat #A11001; INVITROGEN; Dilution 1:200; one-hour incubation at room temperature). Excitation: 488 nm

Bassoon Staining

Primary antibody—Anti-Bassoon (Cat #D63B6; CELL SIGNALING TECHNOLOGY; Dilution 1:150, overnight incubation at 4° C.). Secondary antibody—Goat anti-Rabbit IgG (H+L) Cross-Adsorbed Secondary Antibody, Alexa Fluor 488 (Cat #A11008; INVITROGEN; Dilution 1:200; one-hour incubation at room temperature). Excitation—488 nm

Ribeye Staining

Primary antibody—ANTI-CTBP2 CLONE 16/CTBP2 (RUO) (Cat #612044; BD TRANSDUCTION LABORATORIES; Dilution 1:100, overnight incubation at 4° C.). Secondary antibody—Goat anti-Mouse IgG (H+L) Cross-Adsorbed Secondary Antibody, Alexa Fluor 488 (Cat #A11001; INVITROGEN; Dilution 1:200; one-hour incubation at room temperature). Excitation—488 nm

Blue Opsin Staining

Primary antibody—Anti-Opsin (Cat #AB5407; MILLIPORE; Dilution 1:500, overnight incubation at 4° C.). Secondary antibody—Goat anti-Rabbit IgG (H+L) Cross-Adsorbed Secondary Antibody, Alexa Fluor 488 (Cat #A11008; INVITROGEN; Dilution 1:200; one hour incubation at room temperature). Excitation—488 nm

Nuclear Staining

Reagent—DAPI Fluoromount-G (Cat #0100-20; SOUTHERN BIOTECH; 1 or 2 drops based on area coverage). Excitation—405 nm

Transplanted cells (Pre-labelled with Cell Trace™ Far Red DDAO-SE, LIFE TECHNOLOGIES prior to transplantation). Excitation—633 nm

All markers were examined by fluorescent microscopy and assessed qualitatively by visual examination. A flowchart of rabbit treatment and tissue processing is presented in FIG. 3.

### Results and Discussion

Retinas of rabbits transplanted with AAGP™-treated PPCs and rabbits from the control group (PPC without AAGP treatment) have been analyzed for cell survival. Representative images of rabbit retinas from PPC and PPC+AAGP™-treated groups are shown on FIG. 1. The obtained results indicate that in vitro pre-treatment of PPCs with 4 mg/mL AAGP™ unexpectedly results in a substantial increase of cell survival at 6 months post PPC transplantation into the subretinal area of rabbits with retinal degeneration, despite the absence of immune suppression with cyclosporin past the initial stage of 35 days from start of study.

Additional tests to characterize functional activity and identity of transplanted PPCs were conducted and included fluorescent immunolabeling of Arrestin and Blue Opsin as representative examples of photoreceptor markers and Bassoon and Ribeye as examples of synaptic markers. Immunostaining analysis has been completed for selected animals.

### Conclusion

In conclusion, the study demonstrated that in vitro pre-treatment of PPCs with 4 mg/mL AAGP resulted in a substantial increase of cell survival at 6 months post cell transplantation into the subretinal area of rabbits with retinal degeneration. These results suggest that unexpectedly, the pre-treatment with AAGP™ appears to inhibit immune rejection of the transplanted cells and prevent onset of immune rejection.

### Example 2

### Immunohistochemistry to Detect Cd4+ T Cells

Enucleated mouse eyes with intact conjunctiva were suspended in optimal cutting temperature (OCT) compound and flash frozen in liquid nitrogen. Immunohistochemistry was performed on six-micrometer frozen sections to detect and count the number of cells in conjunctival epithelium that stained positively for CD4 (clone H129.9, 10 μg/mL; BD Biosciences® cat #553647), a biotinylated secondary antibody (BD Pharmingen® cat #559286), and NovaRED® peroxidase (Vector Laboratories® cat #SK-4800). Secondary antibody alone and anti-rat isotype (clone A110-2; BD Biosciences® cat #553992) controls were also examined. Positively stained cells were counted in the GC-rich area of the conjunctiva using image analysis software (Nikon NIS Elements® software) using a 10×objective. T-cell detection and quantification was performed on Arms 1, 2, 4, and 5 only.

Cd4+ T Cell Infiltration

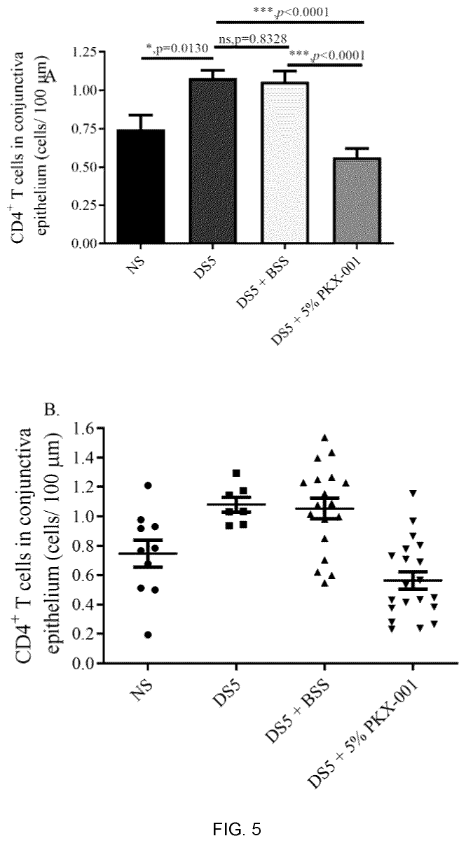

CD4+ T cells infiltration into the conjunctival epithelium is a primary clinical indicator of dry eye disease (DED), which is used here as a proxy to study immune cell infiltration into diseased tissue suffering from retinal degeneration. The presence of CD4+ T cells in the conjunctival epithelium significantly increased in DED-induced mice left untreated (FIGS. 5A and B; *, p=0.0130; Unpaired Student's t-test). Vehicle administration had no effect on reducing T-cell infiltration, whereas bilateral topical administration of 5% PKX-001 significantly reduced T-cell infiltration in DS-induced mice (FIGS. 5A and B; Table 2; ***, p:0.0001; Unpaired Student's t-test).

TABLE 2
T-Cell Density Column Statistics

| | | | | DS5 + 5% |
|---|---|---|---|---|
| | NS | DS5 | DS5 + BSS | PKX-001 |
| Number of values | 10 | 7 | 18 | 20 |
| Minimum | 0.1922 | 0.9365 | 0.5482 | 0.2307 |
| 25% Percentile | 0.5093 | 0.9462 | 0.8143 | 0.3767 |
| Median | 0.7759 | 1.034 | 1.082 | 0.4907 |
| 75% Percentile | 0.9429 | 1.172 | 1.253 | 0.7663 |
| Maximum | 1.208 | 1.293 | 1.538 | 1.152 |
| Mean | 0.747 | 1.079 | 1.054 | 0.5644 |
| Std. Deviation | 0.2905 | 0.1295 | 0.2946 | 0.2614 |
| Std. Error of Mean | 0.09186 | 0.04895 | 0.06944 | 0.05845 |
| Lower 95% Cl of mean | 0.5392 | 0.9595 | 0.9079 | 0.4421 |
| Upper 95% Cl of mean | 0.9548 | 1.199 | 1.201 | 0.6868 |
| Sum | 7.47 | 7.555 | 18.98 | 11.29 |

The results of the CD4+ T cells infiltration assay show that unexpectedly AAGP™ suppresses T-cell response and has a direct impact on the immune system. This ability to act against immune cells was quite surprising and completely unexpected.

### REFERENCES

1) Yanai et al, 2015. Efficient production of photoreceptor precursor cells from human embryonic stem cells. Methods in molecular biology, 1307:357-369.
2) Yanai et al, 2013. Differentiation of human embryonic stem cells using size-controlled embryoid bodies and negative cell selection in the production of photoreceptor precursor cells. Tissue Eng. Part C Methods, 19(10): 755-764.

While preferred embodiments have been described above and illustrated in the accompanying drawings, it will be evident to those skilled in the art that modifications may be made without departing from this disclosure. Such modifications are considered as possible variants comprised in the scope of the disclosure.

1. A method for inhibition or prevention of immune rejection of a graft comprising the step of:
a) contacting an isolated graft prior to a transplantation in a subject in need thereof, with a gem-difluorinated C-glycopeptide compound of general formula I, or a pharmaceutically acceptable base, addition salt with an acid, hydrate or solvate of the compound of general formula I: 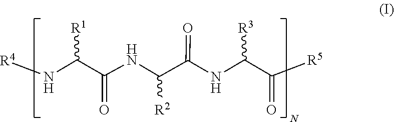
in which:
N is an integer between 1 and 5,
R⁴═H, AA₁, or AA₁-AA₂,
R⁵═OH, AA₁, or AA₁-AA₂,
AA₁ and AA₂ independently represent amino acids with a non-polar side chain and
R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is 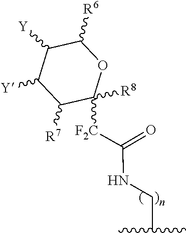
in which:
n is an integer between 3 and 4,
Y, Y′ are independent groups
in which Y, Y′ ═H, OR, N₃, NR′R″, or SR′″,
where R═H, benzyl, trimethylsilyl, tert-butyldimethylsilyl, tert-butyldiphenylsilyl or acetate group,
R′, R″ independently=H, alkyl, allyl, benzyl, tosylate group, C(═O)-alkyl or C(═O)—Bn,
R′″═H, alkyl, or acetate group,
R⁶ is H, CH₃, CH₂OH, CH₂-glycoside group or CH₂—OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function, and
if R¹═R²═H, CH₃, CH₂Ph, CH(CH₃)₂, CH₂CH(CH₃)₂, or CH(CH₃)CH₂CH₃
then R³= 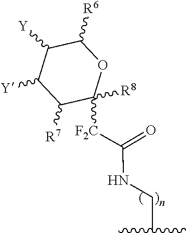
in which: n is an integer between 3 and 4,
Y, Y′ are independent groups
in which Y, Y′ ═H, OR, N₃, NR′R″, or SR′″,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″═H, alkyl, or acetate group,
R⁶ is H, CH₃, CH₂OH, CH₂-glycoside group, or CH₂—OGP in which GP is a protector group selected from alkyl, benzyl, trimethylsilyl, tertbutyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
if R¹═R³═H, CH₃, CH₂Ph, CH(CH₃)₂, CH₂CH(CH₃)₂, or CH(CH₃)CH₂CH₃
then R²= 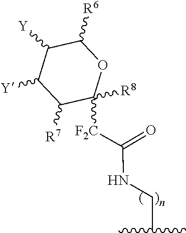
in which: n is an integer between 3 and 4,
Y, Y′ are independent groups
in which Y, Y′ ═H, OR, N₃, NR′R″, or SR′″,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″═H, alkyl, or acetate group,
R⁶ is H, CH₃, CH₂OH, CH₂-glycoside group, or CH₂—OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
if R²═R³═H, CH₃, CH(CH₃)₂, CH₂CH(CH₃)₂, or CH(CH₃)CH₂CH₃
then R¹= 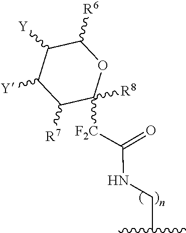
in which: n is an integer between 3 and 4,
Y, Y′ are independent groups
in which Y, Y′ H, OR, N₃, NR′R″, or SR′″,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″═H, alkyl, or acetate group,
R⁶ is H, CH₃, CH₂OH, CH₂-glycoside group, or CH₂—OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tertbutyldimethylsilyl,
tert-butyldiphenylsilyl, or acetate group,
R⁸ is a hydrogen atom H or a free or protected alcohol function
b) transplanting said graft in said subject in need thereof, said subject in need thereof being under an immune suppressant drug therapy to inhibit or prevent immune rejection of said graft;
c) discontinue said immune suppressant drug therapy after a time sufficient for implantation of said graft in said subject in need thereof.

2. The method of claim 1, further comprising step a′) before step a),
a′) isolating said graft.

3. The method of claim 1, wherein said immunosuppressant drug is one of sirolimus, tacrolimus, cyclosporine, everolimus or combinations thereof.

4. The method of claim 1, wherein said subject is a human subject.

5. The method of claim 1, wherein said isolated graft is isolated from a live donor, a cadaveric donor, or combinations thereof.

6. The method of claim 1, wherein the compound of formula I is a compound of formula II: 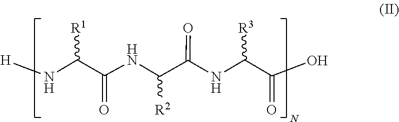
in which: N is an integer between 1 and 5, and
R¹, R², R³ are independent groups in which two of R¹, R² and R³ are selected from H, CH₃ and the remaining R¹, R² and R³ is 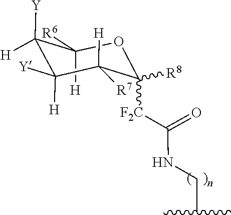
in which: n is an integer between 3 and 4,
Y, Y′ are independent groups
in which Y, Y′ ═H, OR, N₃, NR′R″ or SR′″,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″═H, alkyl, or acetate group,
R⁶ is selected from H, CH₃, CH₂OH, CH₂—OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, NGP′GP″ in which GP′ and GP″ are independently selected from alkyl, benzyl, trimethylsilyl, tert-butyldimethylsilyl,
tert-butyldiphenylsilyl or acetate group,
R⁸ is a hydrogen atom H or a free or protected alcohol function,
and
if R⁸═R²═H or CH₃,
then R³= 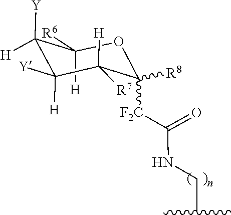
in which: n is an integer between 3 and 4,
Y, Y′ are independent groups
in which Y, Y′ ═H, OR, N₃, NR′R″, or SR′″,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″═H, alkyl, or acetate group,
R⁶ is selected from H, CH₃, CH₂OH, CH₂—OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
if R¹═R³═H or CH₃,
then R²= 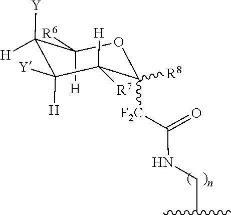
in which: n is an integer between 3 and 4,
Y, Y′ are independent groups
in which Y, Y′ ═H, OR, N₃, NR′R″, SR′″,
where R═H, benzyl, trimethylsilyl, tert-butyldimethylsilyl, tert-butyldiphenylsilyl, or acetate group,
R′, R″ independently=H, alkyl, allyl,
Bn, tosylate, C(═O)-alkyl, or C(═O)—Bn,
R′″═H, alkyl, or acetate group,
R⁶ is selected from H, CH₃, CH₂OH, or CH₂—OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
if R²═R³═H or CH₃,
then R¹= 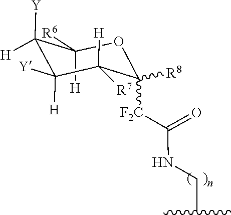
in which: n is an integer between 3 and 4,
Y, Y′ are independent groups
in which Y, Y′ H, OR, N₃, NR′R″, or SR′″,
where R═H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″═H, alkyl, or acetate group,
R⁶ is selected from H, CH₃, CH₂OH, or CH₂—OGP in which GP is a protector group selected from alkyl, benzyl, trimethylsilyl, tert-butyldimethylsilyl, tert-butyldiphenylsilyl, or acetate group,
R⁷═OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom or a free or protected alcohol function.

7. The method of claim 1, wherein said compound of formula I is a compound of formula III:

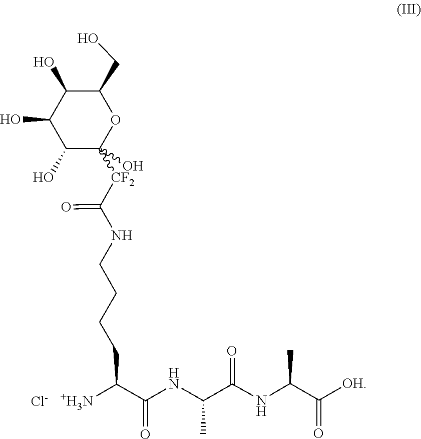

8. The method of claim 1, wherein contacting said isolated graft is with from about from about 0.01 mg/ml to about 5 mg/ml of said compound of formula I, formula II or formula III.

9. The method of claim 8, wherein contacting said isolated graft is with from about 1 mg/ml to about 5 mg/ml of said compound of formula I, formula II or formula III.

10. The method of a claim 1, wherein said isolated graft comprises an organ, a fragment of an organ, a tissue, an isolated cell, or combinations thereof.

11. The method of claim 10, wherein said organ or said fragment of an organ is a liver, a heart, a kidney, a lung, a pancreas, an intestine, a thymus, a uterus, a stomach, a testis, a penis, a hand; and
wherein said tissue is a bone, a bone marrow, a tendon, a cornea, a heart valve, a skin, and a blood vessel; and
wherein said isolated cell is any one of an isolated pancreatic cell, and isolated pancreatic progenitor cell, an adult stem cells, a neurosensory precursor cell, a reticular cell, a glial cell, a neural cell, a cardiac myocyte, an hepatocyte, and an hematopoietic stem cell.

12-13. (canceled)

14. The method of claim 10, wherein said isolated pancreatic cell is an isolated alpha cell, an isolated beta cell, an isolated delta cell, an isolated gamma cell, an epsilon cell, or a combination thereof.

15. (canceled)

16. The method of claim 10, wherein said neurosensory precursor cell is a photoreceptor precursor cell.

17. The method claim 1, wherein said graft is contacted with the compound for about 1 minute to about 1 hour prior to transplantation.

18. The method of claim 1, wherein said graft is contacted with the compound for about 12 hours to about 24 hours prior to transplantation.

19. The method of claim 1, wherein said time sufficient for implantation is about 1 week to about 2 weeks, or at least about 2 weeks.

20. (canceled)

21. The method of claim 1, wherein said subject is under an immune suppressant drug therapy for at least about 1 week prior to transplantation of the graft.

22. The method of claim 11, wherein said organ is a pancreas for the treatment of diabetes.

23. The method of claim 13, wherein said isolated cell is a neurosensory precursor cell for treating a retinal degenerative disease.

24. The method of claim 23, where said retinal degenerative disease is age-related macular degeneration (AMD), retinitis pigmentosa (RP), retinal vasculitis, or sarcoidosis.

25. The method of claim 1, wherein said isolated graft is washed to remove said compound of formula I, II or III.

26-102. (canceled)
